(19) United States
(12) Patent Application Publication
(10) Pub. No.: US 2011/0002863 A1
(43) Pub. Date: Jan. 6, 2011

(54) COSMETIC PRODUCT, USES THEREOF AND MAKE-UP KIT CONTAINING SAID PRODUCT

(75) Inventor: Audrey Ricard, Saint-Maur Des Fosses (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'Oreal, Pairs (FR)

(21) Appl. No.: 12/809,389

(22) PCT Filed: Dec. 19, 2008
(86) PCT No.: PCT/FR08/52390

§ 371 Date: Sep. 15, 2010

Related U.S. Application Data
(60) Provisional application No. 61/006,359, filed on Jan. 8, 2008.

(30) Foreign Application Priority Data
Dec. 20, 2007 (FR) 0760147

(51) Int. Cl. A61K 8/92; A61Q 1/06; A61Q 1/02

(52) U.S. Cl. 424/64; 424/63

(57) ABSTRACT

The invention relates to a cosmetic product for making up keratinous materials, notably the lips, comprising a first composition and a second composition intended to be applied simultaneously,
the first composition containing at least one first oil phase and at least one hydrating agent selected from the polyhydric alcohols, said first composition containing less than 4 wt. % of water, preferably less than 1 wt. % of water relative to the weight of the composition,
and the second composition distinct and different from the first comprising at least one second oil phase and at least one nonvolatile ester oil, said second composition containing less than 50 wt. % of particulate phase, preferably less than 40 wt. %, or more preferably less than 30 wt. % of particulate phase relative to the weight of the composition.

The invention also relates to a method of making up as well as to a make-up kit containing said product. The latter is in particular a lipstick.

The present invention relates to a cosmetic make-up or care product comprising at least two compositions intended to be applied simultaneously on the skin both of the face and of the body, on the lower and upper eyelids, and on the lips. The present invention also relates to a method of making up the face and the body employing these two compositions.

The cosmetic make-up product can be a foundation, a blusher or eye shadow, a concealer, a blush, a lipstick, an eye liner, or a body make-up product.

Make-up artists and consumers demand products that have a range of properties, in particular such as durability and non-transfer, while ensuring a good level of comfort for the user when they are applied on the skin and notably on the lips. Obtaining these various properties means combining different compounds in one composition which, depending on their physical and chemical properties, may be difficult to formulate in one and the same composition, for various reasons.

In particular, it has already been envisaged to incorporate glycerol in lipsticks so as to obtain a composition that is comfortable and hydrating, but the resultant compositions have poor durability and a low level of non-transfer when they are applied on the lips. Moreover, the colours produced are rather unsatisfactory as they are not very bright and not very deep.

One aim of the present invention is to propose make-up that has, at the same time, good durability and a good level of non-transfer. Another aim of the present invention is to propose make-up that has improved properties of comfort, in particular once applied on keratinous materials.

Poor durability over time may be reflected in particular in poor durability of the colour and/or in poor durability of gloss over time. This poor durability may be characterized by a change in colour (colour change, fading) generally as a result of interaction with sebum and/or sweat secreted by the skin in the case of a foundation and blusher or eye shadow, or interaction with saliva in the case of lipsticks, or a decrease in gloss. This obliges the user to reapply the make-up very often, which can mean a loss of time.

The so-called “no transfer” compositions of make-up for the lips and the skin are compositions that have the advantage of forming a deposit that does not transfer, at least partly, on substrates with which they are brought in contact (glass, clothing, cigarettes, fabrics).

The aim of the present invention is to propose a cosmetic product, in particular a make-up product, comprising two compositions that are intended to be applied simultaneously. This cosmetic product can advantageously have good properties of durability, of non-transfer, as well as good properties of comfort.

Unexpectedly and surprisingly, the inventors discovered that production of a cosmetic product comprising on the one hand a composition comprising a hydrating agent selected from the polyhydric alcohols and on the other hand a composition comprising a nonvolatile ester oil proves satisfactory in these terms.

Thus, the products according to the invention make it possible to obtain, if this is desired, a cosmetic rendition that displays, on application and over time, good durability and a good level of non-transfer, as well as being comfortable: it does not dry the skin, the hair or the lips on which it is applied, either during application or in the course of time (little drying, no tightness).

These properties of durability, of non-transfer and of comfort make it a product that is particularly suitable for the production of make-up products for the lips or the skin.

One object of the present invention is therefore a cosmetic product for making up keratinous materials, notably the lips, comprising a first composition and a second composition intended to be applied simultaneously,
the first composition comprising at least one first oil phase and at least one hydrating agent selected from the polyhydric alcohols, said first composition containing less than 10 wt. % of water, preferably less than 4 wt. %, or more preferably less than 0.1 wt. % of water relative to the weight of the composition,
and the second composition, distinct and different from the first, comprising at least one second oil phase and at least one nonvolatile ester oil, said second composition containing less than 50 wt. % of particulate phase, preferably less than 40 wt. %, or more preferably less than 30 wt. % of particulate phase relative to the weight of the composition.

Preferably, the second composition contains less than 4 wt. % of water, preferably less than 1 wt. % of water relative to the weight of the composition

Preferably, the first and/or the second composition only contain(s) traces of water, i.e. less than 0.1% or are completely water-free.

The product of the invention can in particular be a make-up product for the skin, nails or hair.

“Make-up product” means a product containing a colouring agent permitting the deposition of a colour on a keratinous material (the skin or its appendages) of a human being by the application of products such as lipsticks, blusher or eye shadow, eye liners, or foundation, on the keratinous material.

The product according to the invention comprises at least two separate cosmetically acceptable compositions, i.e. which are not mixed with one another prior to application of the product. “Not mixed” means notably that the compositions are not mixed at the macroscopic scale, and the method of manufacture of the product does not include a stage of mixing of the two compositions.

The compositions of the product according to the invention are different. “Different” means that their formulation is not identical.

However, as will be described in more detail, it is possible according to the invention to envisage that the compositions according to the invention are solid and “adjacent” to one another, i.e. cast so that they are in contact with one another, but without being mixed.

The compositions of the product according to the invention are intended to be applied simultaneously and it therefore differs from the “two-part” products described in the prior art, which envisage the successive application of a basecoat and of a topcoat. In the case of two-part products, the two compositions are applied successively, on top of one another.

In contrast, in the case of the product according to the invention, the simultaneous application of the two compositions means that they are mixed more intimately during application.

In particular, this more intimate mixing of the compositions gives an improvement of comfort over time relative to non-transfer or long-lasting compositions whose level of comfort would be improved by the application of a topcoat, which may be lost soon after application, once again leaving a sensation of discomfort for the user.

Advantageously, the compositions of the product according to the invention can be packaged separately or together in one and the same packaging article.

Thus, in the case when the compositions are “packaged separately” in one and the same packaging article, they are not in contact with one another. In the case when they are “packaged together” in one and the same packaging article, the compositions of the product according to the invention are in contact with one another.

The compositions of the product according to the invention can be of different textures and can notably be in the form of a solid, a soft paste or a gel, a mousse, a more or less fluid cream, or a liquid packaged in a tube. In the sense of the invention, “texture” is intended to mean the macroscopic manifestation of the internal structure of a composition.

According to a first embodiment, the two compositions are solid. According to this embodiment, the two compositions can advantageously be packaged together in one and the same packaging article. Preferably, the two compositions are in contact with one another, the solid state of the compositions preventing them from mixing together apart from during use and application of the product, or at least limiting mixing to a possible slight dissolution of the compositions in one another at the boundary between the two compositions.

In particular, in the case of a lipstick product, the compositions are packaged together so as to form a single lipstick.

The make-up product is applied in a single action, like a conventional make-up product, the mixing of the two compositions taking place simultaneously with application on the skin or the lips.

According to a particular embodiment, when the two compositions are in the form of a solid, they can be arranged concentrically in one and the same stick, such as a lipstick, so as to form a single stick, one of the compositions forming the core of the stick and the other forming the cladding of the stick, which surrounds the core composition.

For example, the first composition can optionally be located in the centre of the stick and form its “core”, with the second composition forming the cladding.

One possible method of preparation of such a product is for example by casting the first central composition by means of a mould of suitable diameter. The second composition can then be cast around the first composition, after the latter has cooled, by means of a second mould suitable for forming the “cladding” of the stick.

According to a second embodiment, the two compositions are liquid.

Preferably, when the two compositions are in the form of liquids, they are packaged separately in one and the same container (packaging article), i.e. separated by a wall for example, to prevent them mixing together. According to this embodiment, the product comprises an applicator permitting simultaneous application of the two compositions on said keratinous materials, mixing taking place simultaneously with application on the skin or the lips.

In the case of a make-up product for the lips, the product according to the invention thus preferably forms, in its method of use, a lipstick that is applied in a single action.

Such a device permitting the simultaneous application of two liquid compositions is described for example in application EP 1656853.

According to a preferred embodiment, when the two compositions are in liquid form, they are advantageously contained in two tubes arranged concentrically relative to one another, i.e. one of the two tubes is housed inside the other tube.

Preferably, when at least one of the compositions is in liquid form, the compositions are packaged separately in one and the same container, to prevent them mixing together. Preferably, when at least one of the two compositions is in liquid form, they are packaged separately in one and the same container (or packaging article) so that they do not mix together and so that mixing only occurs during application and essentially only involves the amount of each composition that is applied, i.e. concomitantly with application, the latter being applied simultaneously during use of the cosmetic product by the user.

The terms “solid” and “liquid” characterize the state of a composition at room temperature (25° C.) and at atmospheric pressure (760 mmHg).

The texture of a composition can be measured by its hardness and/or its viscosity.

### Protocol for Measurement of Hardness:

Measurement is carried out according to the following protocol:

A sample of the composition in question is cast hot in a lipstick mould with diameter of 12.7 mm. The mould is then cooled in a freezer for about an hour. The lipstick is then stored at 20° C.

The hardness of the samples is measured after waiting 24 hours.

The hardness of the samples of compositions of the invention, expressed in grams, is measured using a DFGS2 dynamometer marketed by the company INDELCO-CHATILLON.

The hardness corresponds to the maximum shearing force exerted by a rigid tungsten wire with a diameter of 250 μm advancing at a speed of 100 mm/min. The technique described above is usually called the “butter cutter wire” method.

“Solid” composition means a composition whose hardness is between 30 and 300 g, or even from 50 to 200 g.

### Protocol for Measurement of Viscosity

The viscosity of a composition of the invention can be measured as follows.

The measurements can be carried out using an imposed-stress rheometer, RS 75 from the company ThermoRheo, equipped with a thermostatic bath and a stainless steel spindle of cone and plate geometry, the cone having a diameter of 35 mm, an angle of 2° and a gap (distance between the bottom plate—called the stator plate—on which the composition is deposited and the top plate—called the rotor plate) of 0.3 mm.

The measurements are carried out at 25° C.±0.5° C.

Analysis in equilibrium flow conditions consists of submitting a sample of the composition, starting from a given moment, to an instantaneous shear stress τ (shearing), which is kept constant for a time t (waiting time, selected so that a steady state is reached, t=30s).

According to a preferred embodiment, the first and second compositions of the product according to the invention have similar textures.

Preferably, when the two compositions are solid, they have similar hardness and when the two compositions are liquid, they are formulated so that they have similar viscosity.

Preferably, the compositions of the product according to the invention are formulated so that they are compatible, i.e. readily miscible with one another.

Advantageously, the compositions can be arranged concentrically relative to one another. According to this embodiment, the first composition can be arranged on the inside or on the outside, relative to the second composition.

According to a particularly preferred embodiment, the first composition comprising the hydrating agent is arranged inside the second composition. According to this embodiment, the two compositions are directly in contact with one another on the entire periphery of the first composition in the case when they are both solid.

Preferably, the second composition contains less than 4 wt. % of water, preferably less than 1 wt. % of water relative to the weight of the composition.

The invention also relates to a make-up kit containing a cosmetic make-up product as defined previously, in which the different compositions are packaged separately and are advantageously accompanied by suitable means of application. These means can be brushes of various kinds, pens, pencils, felt-tip pens, pens, sponges, tubes and/or foam tips.

The product according to the invention can be applied on the skin both of the face and of the scalp and of the body, the lips, the inside of the bottom eyelids, and the appendages such as the nails, eyelashes, hair, eyebrows, or even body hair. This make-up can also be applied on make-up accessories such as false nails, false eyelashes, wigs or even patches adhering to the skin or the lips (of the beauty spot type).

The invention also relates to the cosmetic use of the cosmetic product defined above for improving the properties of durability, of non-transfer and of comfort of make-up on the skin and/or the lips and/or the integumentary appendages.

The invention finally relates to the use of a first composition comprising at least one oil phase and at least one hydrating agent selected from the polyhydric alcohols, and of a second composition comprising at least one oil phase and at least one nonvolatile ester oil, said first composition containing less than 4 wt. % of water, preferably less than 1 wt. % of water relative to the weight of the composition and said second composition containing less than 50 wt. % of particulate phase, preferably less than wt. %, or more preferably less than 30 wt. % of particulate phase relative to the weight of the composition, for endowing the skin and/or the lips and/or the integumentary appendages with a comfortable cosmetic rendition, having a good durability and a good level of non-transfer, as well as good properties of comfort.

Hydrating Agent

The first composition according to the invention comprises at least one hydrating agent selected from the polyhydric alcohols, preferably C₂-C₈, and more preferably C₃-C₆.

Preferably, the polyhydric alcohols that can be used are selected from glycerol, propylene glycol, 1,3-butylene glycol, dipropylene glycol, diglycerol, and mixtures thereof.

Preferably, the hydrating agent present in the first composition is glycerol.

Preferably, the amount of hydrating agent in the first composition is in the range for example from 0.001 to 30 wt. %, preferably from 0.01 to 20 wt. %, or better still from 0.01 to 10 wt. % relative to the total weight of the composition.

Preferably, the second composition comprises less than 3 wt. % of hydrating agent selected from the polyhydric alcohols, relative to the weight of the composition.

More preferably, the second composition is completely free of hydrating agent selected from the polyhydric alcohols.

### Colorants

The first and/or the second composition of the cosmetic product according to the invention contains at least one colouring agent (also called “colorant”) which can notably be selected from water-soluble or fat-soluble dyes, pigments, nacres, glitter and mixtures thereof.

Preferably, the colorants are present, in the first and/or in the second composition, at a content in the range from 0.01 to 50 wt. %, relative to the weight of the composition, preferably from 0.01 to 30 wt. %.

“Pigments” are to be understood as white or coloured particles, mineral or organic, insoluble in an aqueous solution, intended to colour and/or opacify the resultant film.

The pigments can be present at a rate from 0.01 to 20 wt. %, notably from 0.01 to 15 wt. %, and in particular from 0.02 to 10 wt. %, relative to the total weight of the cosmetic composition.

Goniochromatic Colouring Agents

The first composition and/or the second composition can include at least one goniochromatic colouring agent, which can have magnetic properties if required.

“Goniochromatic colouring agent” means, in the sense of the present invention, a colouring agent that makes it possible to obtain, when the composition is spread on a substrate, a colour path in the a*b* plane of the CIE 1976 colorimetric space which corresponds to a change Dh in hue angle h of at least 20° when the angle of observation relative to the normal is varied between 0° and 80°, for an angle of incidence of the light of 45°.

The colour path can be measured for example using a spectrogonioreflectometer made by INSTRUMENT SYSTEMS with the reference GON 360 GONIOMETER, after the first composition has been spread out in the fluid state with a thickness of 300 μm by means of an automatic spreader on a contrast card made by ERICHSEN with the reference Type 24/5, measurement being effected against the black background of the card.

The goniochromatic colouring agent can be selected for example from multilayer interference structures and liquid-crystal colouring agents.

In the case of a multilayer structure, it can for example comprise at least two layers, each layer being made for example from at least one material selected from the group comprising the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and combinations thereof.

The multilayer structure may or may not have, relative to a central layer, symmetry at the level of the chemical nature of the stacked layers. Different effects are obtained, depending on the thickness and the nature of the various layers.

Examples of symmetric multilayer interference structures are for example the following structures: Fe₂O₃/SiO₂/Fe₂O₃/SiO₂/Fe₂O₃, a pigment having this structure being marketed under the name SICOPEARL by the company BASF; MoS₂/SiO₂/mica oxide/SiO₂/MoS₂; Fe₂O₃/SiO₂/mica oxide/SiO₂/Fe₂O₃; TiO₂/SiO₂/TiO₂ and TiO₂/Al₂O₃/TiO₂, pigments having these structures being marketed under the name XIRONA by the company MERCK (Darmstadt).

The liquid-crystal colouring agents comprise for example silicones or cellulose ethers on which mesomorphic groups are grafted. As liquid-crystal goniochromatic particles, it is for example possible to use those sold by the company CHENIX as well as those marketed under the name HELICONE® HC by the company WACKER.

As goniochromatic colouring agent, it is also possible to use certain nacres, effect pigments on a synthetic substrate, notably a substrate such as alumina, silica, borosilicate, iron oxide, aluminium, or holographic interference glitter obtained from a polyterephthalate film.

The ratio of the proportion by weight of the magnetic pigments to the proportion of goniochromatic colouring agent is for example between ¼ and 4, being for example between % and 2, for example close to 1.

The material can moreover contain dispersed goniochromatic fibres. Said fibres can have a length of less than 80 μm for example.

Diffracting Pigments

The first composition and/or the second composition of the product according to the invention can include at least one diffracting pigment, which can have magnetic properties if required.

“Diffracting pigment” means, in the sense of the present invention, a pigment capable of producing a colour change according to the angle of observation when lit by white light, owing to the presence of a structure that diffracts the light.

A diffracting pigment can include a diffraction grating, capable for example of diffracting an incident ray of monochromatic light in defined directions.

The diffraction grating can include a periodic unit, notably a line, the distance between two adjacent units being of the same order of magnitude as the wavelength of the incident light.

When the incident light is polychromatic, the diffraction grating will separate the various spectral components of the light and produce a rainbow effect.

Regarding the structure of the diffracting pigments, it may be useful to refer to the article “Pigments Exhibiting Diffractive Effects” by Alberto Argoitia and Matt Witzman, 2002, Society of Vacuum Coaters, 45^{th} Annual Technical Conference Proceedings 2002.

The diffracting pigment can be made with units having different profiles, notably triangular, symmetric or asymmetric, with gaps, of constant or variable width, sinusoidal.

The spatial frequency of the grating and the depth of the units will be selected in relation to the degree of separation of the various orders desired. The frequency can vary for example between 500 and 3000 lines per mm.

Preferably, the particles of the diffracting pigment each have a flattened shape, and notably are in the form of small plates.

One and the same pigment particle can have two crossed diffraction gratings, perpendicular or otherwise.

The diffracting pigment can have a multilayer structure comprising a layer of a reflective material, covered at least on one side with a layer of a dielectric material. The latter can endow the diffracting pigment with better rigidity and durability. The dielectric material can then be selected for example from the following materials: MgF₂, SiO₂, Al₂O₃, AlF₃, CeF₃, LaF₃, NdF₃, SmF₂, BaF₂, CaF₂, LiF and combinations thereof. The reflective material can be selected for example from metals and metal alloys as well as from non-metallic reflective materials. Among the metals that can be used, we may mention Al, Ag, Cu, Au, Pt, Sn, Ti, Pd, Ni, Co, Rd, Nb, Cr and materials, combinations or alloys thereof. Said reflective material can, on its own, constitute the diffracting pigment, which will then be a monolayer.

As a variant, the diffracting pigment can comprise a multilayer structure having a core of a dielectric material covered with a reflective layer on at least one side, or even encapsulating the core completely. A layer of a dielectric material can also cover the reflective layer or layers. The dielectric material used is then preferably inorganic, and can be selected for example from metal fluorides, metal oxides, metal sulphides, metal nitrides, metal carbides and combinations thereof. The dielectric material can be in the crystalline, semi-crystalline or amorphous state. The dielectric material, in this configuration, can for example be selected from the following materials: MgF₂, SiO, SiO₂, Al₂O₃, TiO₂, WO, AlN, BN, B₄C, WC, TiC, TiN, N₄Si₃, ZnS, glass particles, carbon materials of the diamond type and combinations thereof.

As a variant, the diffracting pigment can be composed of a preformed dielectric or ceramic material such as a natural lamellar mineral, for example mica, peroskovite or talc, or synthetic lamellae formed from glass, alumina, SiO₂, carbon, an iron oxide/mica, mica covered with BN, BC, graphite, bismuth oxychloride, and combinations thereof.

Instead of a layer of a dielectric material, other materials improving the mechanical properties may be suitable. Such materials can comprise silicone, metal silicides, semiconductor materials formed from elements of groups III, IV and V, metals having a cubic centred crystalline structure, cermet compositions or materials, semiconducting glasses, and various combinations thereof.

The diffracting pigment used can notably be selected from those described in US patent application US 2003/0031870 published 13 Feb. 2003.

A diffracting pigment can comprise for example the following structure: MgF₂/Al/MgF₂, a diffracting pigment having this structure being marketed under the name SPECTRAFLAIR 1400 Pigment Silver by the company FLEX PRODUCTS, or SPECTRAFLAIR 1400 Pigment Silver FG. The proportion by weight of MgF₂ can be between 80 and 95% of the total weight of the pigment.

The amount of diffracting pigment can vary, by weight relative to the total weight of the first composition, for example from 0.1 to 5%.

The size of the diffracting pigment can be for example between 5 and 200 μm, preferably between 5 and 100 μm, for example between 5 and 30 μm.

The thickness of the particles of diffracting pigment can be less than or equal to 3 μm, preferably 2 μm, for example of the order of 1 μm.

Reflective Particles

The first composition and/or the second composition of the product according to the invention can comprise for example reflective particles, notably glitter, among others, magnetic or non-magnetic.

“Reflective particles” means, in the sense of the present invention, particles whose size, structure, notably the thickness of the layer or layers of which it is constituted and their physical and chemical natures, and the surface condition, permit them to reflect incident light. Said reflection can, in certain cases, be of sufficient intensity to create, at the surface of the composition or of the mixture, when the latter is applied on the substrate to be made up, superglossy points that are visible to the naked eye, i.e. points that are more luminous, which contrast with their surroundings and appear to shine.

The reflective particles can be selected so as not to alter significantly the colouring effect produced by the colouring agents with which they are combined, and more particularly so as to optimize said effect in terms of colour rendition. They can more particularly possess a yellow, pink, red, bronze, orange, brown, gold and/or copper colour or sheen.

The reflective particles can be present in the first composition at a content in the range from 0.5 to 60% relative to the total weight of the first composition, notably from 1 to 30 wt. %, in particular from 3 to 10 wt. %.

These particles can be in various forms, notably in the form of small plates or can be globular, in particular spherical.

The reflective particles, whatever their shape, may or may not have a multilayer structure, and in the case of a multilayer structure can have for example at least one layer of uniform thickness, notably of a reflective material.

When the reflective particles do not have a multilayer structure, they can be composed for example of metal oxides, notably oxides of titanium or of iron obtained by synthesis.

When the reflective particles have a multilayer structure, they can for example comprise a natural or synthetic substrate, notably a synthetic substrate at least partially coated with at least one layer of a reflective material notably of at least one metal or metallic material. The substrate can be of a single material, of multiple materials, organic and/or inorganic.

More particularly, it can be selected from glasses, ceramics, graphite, metal oxides, aluminas, silicas, silicates, notably aluminosilicates and borosilicates, synthetic mica and mixtures thereof, this list not being exhaustive.

The reflective material can comprise a layer of metal or of a metallic material.

Reflective particles are described notably in documents

### JP-A-09188830, JP-A-10158450, JP-A-10158541, JP-A-07258460 and JP-A-05017710.

As further examples of reflective particles having a mineral substrate coated with a layer of metal, we may also mention particles having a substrate of silver-coated borosilicate, also called “white nacres”.

Particles with a substrate of silver-coated glass, in the form of small plates, are sold under the name MICROGLASS METASHINE REFSX 2025 PS by the company TOYAL. Particles with a substrate of glass coated with nickel/chromium/molybdenum alloy are sold under the name CRYSTAL STAR GF 550, GF 2525 by this same company.

The reflective particles, regardless of their shape, can also be selected from particles with a synthetic substrate coated at least partially with at least one layer of at least one metallic material, notably a metal oxide, selected for example from oxides of titanium, notably TiO₂, of iron notably Fe₂O₃, of tin, of chromium, barium sulphate and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂ and mixtures or alloys thereof.

As examples of said particles, we may mention for example particles having a substrate of synthetic mica coated with titanium dioxide, or particles of glass coated either with brown iron oxide, oxide of titanium, oxide of tin or with a mixture thereof such as those sold under the trade name REFLECKS® by the company ENGELHARD.

Nacres

The first composition and/or the second composition of the product according to the invention can comprise at least one nacre, magnetic or non-magnetic.

“Nacre” is to be understood as meaning coloured particles of any shape, iridescent or non-iridescent, notably produced in the shell of certain molluscs or else synthesized, and which have a colour effect by optical interference.

Nacres can be selected from the nacreous pigments such as titanium mica covered with an iron oxide, mica covered with bismuth oxychloride, titanium mica covered with chromium oxide, titanium mica covered with an organic dye notably of the aforementioned type as well as nacreous pigments based on bismuth oxychloride. They may also be mica particles, on the surface of which at least two successive layers of metal oxides and/or of organic dyes are superposed.

We may also mention, as examples of nacres, natural mica covered with titanium oxide, iron oxide, natural pigment or bismuth oxychloride.

Among the nacres available on the market, we may mention the nacres TIMICA, FLAMENCO and DUOCHROME (based on mica) marketed by the company ENGELHARD, the TIMIRON nacres marketed by the company MERCK, the PRESTIGE mica-based nacres marketed by the company ECKART and the SUNSHINE nacres based on synthetic mica marketed by the company SUN CHEMICAL.

The nacres can more particularly possess a yellow, pink, red, bronze, orange, brown, gold and/or copper colour or reflection.

By way of illustration of the nacres that can be incorporated in the first composition, we may notably mention the nacres of gold colour notably marketed by the company ENGELHARD under the name Brillant gold 212G (Timica), Gold 222C (Cloisonne), Sparkle gold (Timica), Gold 4504 (Chromalite) and Monarch gold 233X (Cloisonne); the bronze nacres notably marketed by the company MERCK under the name Bronze fine (17384) (Colorona) and Bronze (17353) (Colorona) and by the company ENGELHARD under the name Super bronze (Cloisonne); the orange nacres notably marketed by the company ENGELHARD under the name Orange 363C (Cloisonne) and Orange MCR 101 (Cosmica) and by the company MERCK under the name Passion orange (Colorona) and Matte orange (17449) (Microna); the brown coloured nacres notably marketed by the company ENGELHARD under the name Nu-antique copper 340XB (Cloisonne) and Brown CL4509 (Chromalite); the nacres with a copper sheen notably marketed by the company ENGELHARD under the name Copper 340A (Timica); the nacres with a red sheen notably marketed by the company MERCK under the name Sienna fine (17386) (Colorona); the nacres with a yellow sheen notably marketed by the company ENGELHARD under the name Yellow (4502) (Chromalite); the red coloured nacres with a gold sheen notably marketed by the company ENGELHARD under the name Sunstone G012 (Gemtone); the pink nacres notably marketed by the company ENGELHARD under the name Tan opale G005 (Gemtone); the black nacres with a gold sheen notably marketed by the company ENGELHARD under the name Nu antique bronze 240 AB (Timica), the blue nacres notably marketed by the company MERCK under the name Matte blue (17433) (Microna), the white nacres with a silver sheen notably marketed by the company MERCK under the name Xirona Silver and the orange-pink-green-golden nacres notably marketed by the company MERCK under the name Indian summer (Xirona) and mixtures thereof.

Organic Dyes or Pigments and Lakes

The first composition and/or the second composition can additionally comprise organic dyes or pigments.

The dyes can be fat-soluble or water-soluble.

The fat-soluble dyes are for example Sudan red, DC Red 17, DC Green β-carotene, soya oil, Sudan brown, DC Yellow 11, DC Violet 2, DC orange 5, quinoline yellow.

The water-soluble dyes are for example beetroot juice and methylene blue.

The dyes can for example represent from 0.1 to 20% of the weight of the first or of the second composition, or even from 0.1 to 6%, when present.

The organic lakes are organic pigments formed by a dye fixed on a substrate.

The lakes, which are also called organic pigments, can be selected from the following materials and mixtures thereof:
cochineal carmine
the organic pigments of azo, anthraquinone, indigoid, xanthene, pyrene, quinoline dyes, triphenylmethane, fluorane. Among the organic pigments, we may notably mention those known by the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.
the organic lakes can be insoluble salts of sodium, of potassium, of calcium, of barium, of aluminium, of zirconium, of strontium, of titanium, acid dyes such as the azo, anthraquinone, indigoid, xanthene, pyrene, quinoline dyes, triphenylmethane, fluorane, and said dyes can have at least one carboxylic or sulphonic acid group.

The organic lakes can also be carried by an organic carrier such as rosin or aluminium benzoate, for example.

Among the organic lakes, we may in particular mention those known by the following names: D&C Red No. 2 Aluminium lake, D&C Red No. 3 Aluminium lake, D&C Red No. 4 Aluminium lake, D&C Red No. 6 Aluminium lake, D&C Red No. 6 Barium lake, D&C Red No. 6 Barium/Strontium lake, D&C Red No. 6 Strontium lake, D&C Red No. 6 Potassium lake, D&C Red No. 7 Aluminium lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminium lake, D&C Red No. 9 Barium lake, D&C Red No. 9 Barium/Strontium lake, D&C Red No. 9 Zirconium lake, D&C Red No. 10 Sodium lake, D&C Red No. 19 Aluminium lake, D&C Red No. 19 Barium lake, D&C Red No. 19 Zirconium lake, D&C Red No. 21 Aluminium lake, D&C Red No. 21 Zirconium lake, D&C Red No. 22 Aluminium lake, D&C Red No. 27 Aluminium lake, D&C Red No. 27 Aluminium/Titanium/Zirconium lake, D&C Red No. 27 Barium lake, D&C Red No. 27 Calcium lake, D&C Red No. 27 Zirconium lake, D&C Red No. 28 Aluminium lake, D&C Red No. 30 lake, D&C Red No. 31 Calcium lake, D&C Red No. 33 Aluminium lake, D&C Red No. 34 Calcium lake, D&C Red No. 36 lake, D&C Red No. 40 Aluminium lake, D&C Blue No. 1 Aluminium lake, D&C Green No. 3 Aluminium lake, D&C Orange No. 4 Aluminium lake, D&C Orange No. 5 Aluminium lake, D&C Orange No. 5 Zirconium lake, D&C Orange No. 10 Aluminium lake, D&C Orange No. 17 Barium lake, D&C Yellow No. 5 Aluminium lake, D&C Yellow No. 5 Zirconium lake, D&C Yellow No. 6 Aluminium lake, D&C Yellow No. 7 Zirconium lake, D&C Yellow No. 10 Aluminium lake, FD&C Blue No. 1 Aluminium lake, FD&C Red No. 4 Aluminium lake, FD&C Red No. 40 Aluminium lake, FD&C Yellow No. 5 Aluminium lake, FD&C Yellow No. 6 Aluminium lake.

The chemical materials corresponding to each of the aforementioned organic dyes are mentioned in the work “International Cosmetic Ingredient Dictionary and Handbook”, Edition 1997, pages 371 to 386 and 524 to 528, published by “The Cosmetic, Toiletry, and Fragrance Association”, the contents of which are incorporated in the present application by reference.

Composite Pigments

The first composition and/or the second composition can additionally comprise composite pigments.

The composite pigment can be composed notably of particles comprising:
a magnetic or non-magnetic inorganic core,
at least one at least partial coating of at least one organic colorant.

At least one binder can advantageously contribute to fixation of the organic colorant on the inorganic core.

The particles of composite pigment can have various shapes. Said particles can notably be in the form of small plates or can be globular, in particular spherical, and can be hollow or solid. “In the form of small plates” means particles whose ratio of the largest dimension to the thickness is greater than or equal to 5.

A composite pigment can have for example a specific surface between 1 and 1000 m²/g, notably between about 10 and 600 m²/g, and in particular between about 20 and 400 m²/g. The specific surface is the value measured by the BET method.

The inorganic core of the composite pigment can be of any shape suitable for the fixation of particles of organic colorant, for example spherical, globular, granular, polyhedral, acicular, fusiform, flattened in the form of flakes, like a grain of rice, scales, as well as a combination of these shapes, this list not being exhaustive.

The ratio of the largest dimension of the core to its smallest dimension can be between 1 and 50.

The inorganic core can have a size between about 1 nm and about 100 nm, or even between about 5 nm and about 75 nm, for example between about 10 nm and about 50 nm.

The inorganic core can be made of a material selected from the non-exhaustive list comprising metal salts and metal oxides, notably oxides of titanium, of zirconium, of cerium, of zinc, of iron, of ferric blue, of aluminium and of chromium, aluminas, glasses, ceramics, graphite, silicas, silicates, notably aluminosilicates and borosilicates, synthetic mica, and mixtures thereof.

The oxides of titanium, notably TiO₂, of iron, notably Fe₂O₃, of cerium, of zinc and of aluminium, the silicates, notably the aluminosilicates and the borosilicates, are quite particularly suitable.

The inorganic core can have a specific surface, measured by the BET method, for example between about 1 m²/g and about 1000 m²/g, preferably between about 10 m²/g and about 600 m²/g, for example between about 20 m²/g and about 400 m²/g.

The inorganic core can be coloured, if required.

The organic colorant can be as defined above.

The binder of the composite pigment can be of any type provided it enables the organic colorant to adhere to the surface of the inorganic core.

The binder can notably be selected from a non-exhaustive list comprising silicone materials, polymeric, oligomeric or similar materials, and in particular from organosilanes, fluoroalkylated organosilanes and polysiloxanes, for example polymethylhydrogen-siloxane, as well as various couplers, such as couplers based on silanes, titanates, aluminates, zirconates and mixtures thereof.

The colouring agent present in the second composition and which may be present in the first composition can comprise a photochromic colorant or photochromic agent.

Photochromic Agents

Generally, a photochromic colouring agent is a colouring agent having the property of changing hue when it is lit by ultraviolet light and of regaining its original colour when it is no longer lit by said light or of changing from a non-coloured state to a coloured state and vice versa. In other words, such an agent has different hues according to whether it is lit by light containing a certain amount of UV radiation such as in sunlight or by artificial light.

It will be useful to refer to the examples of photochromic agents described in EP 1 410 786.

Thermochromic Agents

It is possible for example to use the thermochromic agent marketed under the reference KROMAFAST YELLOW 5GX 02—by the company KROMACHEM LTD.

Other Colouring Agents

The second composition and/or the first composition can additionally comprise piezochromic, notably tribochromic, or solvatochromic compounds.

### Particulate Phase

The second composition of the product according to the invention contains less than 50 wt. % of particulate phase, preferably less than 40 wt. %, or more preferably less than 30 wt. % of particulate phase relative to the weight of the composition.

By particulate phase we mean the pigments and nacres as defined previously, as well as the fillers that are defined below.

Preferably, the first composition of the product according to the invention contains less than 50 wt. % of particulate phase, preferably less than 40 wt. %, or more preferably less than 30 wt. % of particulate phase relative to the weight of the composition.

### Oil Phase:

The first and second compositions of the product according to the invention each comprise an oil phase called, for greater clarity, first oil phase and second oil phase, respectively.

The first and/or second oil phase of the compositions of the product according to the invention is in particular a liquid oil phase based on at least one oil.

### 1. Liquid Oil Phase

### Nonvolatile Oils

The first and/or the second oil phase of the first and second compositions of the product according to the invention can comprise at least one nonvolatile oil. The nonvolatile oils can be hydrocarbon oils and/or silicone oils and/or fluorinated oils.

“Oil” means a non-aqueous compound, immiscible with water, liquid at room temperature (25° C.) and atmospheric pressure (760 mmHg).

“Nonvolatile oil” means an oil that remains on keratinous materials at room temperature and atmospheric pressure for at least several hours and notably has a vapour pressure of less than 10⁻³ mmHg (0.13 Pa).

These oils can be of vegetable, mineral or synthetic origin.

“Hydrocarbon oil” means an oil formed essentially, or even constituted, of carbon atoms and of hydrogen, and optionally of oxygen and nitrogen atoms, and not containing silicon or fluorine atoms. It can contain alcohol, ester, ether, carboxylic acid, amine and/or amide groups.

“Silicone oil” means an oil comprising at least one silicon atom, and notably comprising Si—O groups.

As nonvolatile hydrocarbon oil, we may notably mention:
hydrocarbon oils of vegetable origin such as triglycerides constituted of esters of fatty acids and of glycerol, where the fatty acids can have various chain lengths from C₄ to C₂₄, which can be linear or branched, saturated or unsaturated, such as triglycerides of heptanoic acid and octanoic acid; these oils are notably wheatgerm oil, sunflower oil, grapeseed oil, sesame oil, maize oil, apricot oil, castor oil, shea oil, avocado oil, olive oil, soya oil, sweet almond oil, palm oil, colza oil, cotton oil, hazelnut oil, macadamia oil, jojoba oil, alfalfa oil, poppy oil, Chinese okra oil, sesame oil, cucurbit oil, colza oil, blackcurrant oil, evening primrose oil, millet oil, barley oil, quinoa oil, rye oil, safflower oil, candlenut oil, passionflower oil, musk rose oil; or triglycerides of caprylic/capric acids such as those sold by the company Stéarineries Dubois or those sold under the names “Miglyol 810®”, “812®” and “818®” by the company Dynamit Nobel,
synthetic ethers having from 10 to 40 carbon atoms;
linear or branched hydrocarbons, of mineral or synthetic origin such as paraffin oil or its derivatives, petroleum jelly, polydecenes, hydrogenated polyisobutylene such as Parleam® marketed by the company NIPPON OIL FATS, squalane, and mixtures thereof;
esters of fatty acid and of a polyol, said fatty acid having in particular from 6 to 22 carbon atoms, and notably octanoic acid, heptanoic acid, lanolin acid, oleic acid, lauric acid, stearic acid, as ester we may mention propylene glycol dioctanoate, propylene glycol monoisostearate, poly-glyceryl 2-diisostearate, neopentylglycol diheptanoate,
synthetic esters such as oils of formula R₁COOR₂ in which R₁ represents the residue of a linear or branched fatty acid having from 1 to 40 carbon atoms and R₂ represents a hydrocarbon chain notably branched containing from 1 to 40 carbon atoms provided that R₁+R₂ is ≧11, for example Purcellin oil (cetostearyl octanoate), isononyl isononanoate, benzoate of C₁₂ to C₁₅ alcohol, ethyl 2-hexyl palmitate, octyl-2 dodecyl stearate, octyl-2 dodecyl erucate, isostearyl isostearate, octyl-2 dodecyl benzoate, octanoates, decanoates or ricinoleates of alcohols or of polyalcohols, isopropyl myristate, isopropyl palmitate, butyl stearate, hexyl laurate, diisopropyl adipate, 2-ethylhexyl palmitate, 2-hexyldecyl laurate, 2-octyldecyl palmitate, 2-octyldodecyl myristate, 2-diethylhexyl succinate, diisostearyl malate, isodecyl neopentanoate;
hydroxylated esters such as isostearyl lactate, octyl hydroxystearate, octyldodecyl hydroxystearate, diisostearylmalate, triisocetyl citrate, glycerol or diglycerol triisostearate; diethylene glycol diisononanoate; and
fatty alcohols that are liquid at room temperature with a branched carbon chain and/or unsaturated having from 8 to 26 carbon atoms such as oleic alcohol, linoleic or linolenic alcohol, isostearic alcohol or octyl dodecanol;
C₈-C₂₆ higher fatty acids such as oleic acid, linoleic acid, linolenic acid, or isostearic acid;
and mixtures thereof.

The nonvolatile silicone oils usable in the first and/or the second composition according to the invention can be phenylated silicone oils (phenylated silicone means an organopolysiloxane substituted with at least one phenyl group), nonvolatile polydimethylsiloxanes (PDMS), polydimethylsiloxanes bearing alkyl or alkoxy groups, pendant and/or at the end of the silicone chain, each of said groups having from 2 to 24 carbon atoms.

In particular, the second composition of the product according to the invention comprises at least one nonvolatile ester oil.

Preferably, the nonvolatile ester oil is a hydrocarbon oil.

Advantageously, the hydrocarbon ester oil has a viscosity of less than 15 cSt.

Preferably, the nonvolatile ester oil of the second composition is selected from the synthetic esters such as the oils of formula R₁COOR₂ in which R₁ represents the residue of a linear or branched fatty acid having from 1 to 40 carbon atoms and R₂ represents a hydrocarbon chain notably branched containing from 1 to 40 carbon atoms provided that R₁+R₂ is ≧11, for example Purcellin oil (cetostearyl octanoate), isononyl isononanoate, benzoate of C₁₂ to C₁₅ alcohol, ethyl 2-hexyl palmitate, octyl-2 dodecyl stearate, octyl-2 dodecyl erucate, isostearyl isostearate, octyl-2 dodecyl benzoate, octanoates, decanoates or ricinoleates of alcohols or of polyalcohols, isopropyl myristate, isopropyl palmitate, butyl stearate, hexyl laurate, diisopropyl adipate, 2-ethylhexyl palmitate, 2-hexyldecyl laurate, 2-octyldecyl palmitate, 2-octyldodecyl myristate, 2-diethylhexyl succinate, diisostearyl malate, isodecyl neopentanoate.

Preferably, the nonvolatile ester oil is selected from isopropyl isostearate, isononyl isononanoate, isostearyl heptanoate.

Preferably, the first composition of the product according to the invention also comprises at least one nonvolatile oil, which can also be an ester oil, such as a hydrocarbon ester oil, such as mentioned previously.

Advantageously, the oil phase of the first and/or of the second oil phase of the compositions of the product according to the invention comprises from 0.1 to 60 wt. % nonvolatile ester oil or oils, preferably from 5 to 50 wt. % relative to the total weight of the first or of the second composition, as appropriate.

The second composition of the product according to the invention can also comprise an additional nonvolatile oil, different from the ester oil.

The nonvolatile oil can be present at a content in the range from 0.1 to 80 wt. %, preferably from 1 to 60 wt. %, better still from 5 to 50 wt. % relative to the total weight of the composition.

### Gloss Oil

According to one embodiment of the invention, the nonvolatile oil can be a gloss oil. The first and/or the second oil phase of the compositions of the product according to the invention can thus comprise at least one gloss oil.

In particular, according to one embodiment, one of the compositions can comprise a gloss oil in a sufficient amount to provide at least one aspect of the performance of make-up of the gloss type.

Preferably, the second composition comprises at least one gloss oil.

Preferably, the gloss oil or gloss oils represent from 0.1 to 80%, preferably from 1 to 60%, and more preferably from 5 to 50% of the total weight of the first and/or of the second composition.

The gloss oil preferably has a high molecular weight in the range from 650 to 10 000 g/mol, and preferably between 750 and 7500 g/mol.

The gloss oil to be used in the present invention can be selected from:
lipophilic polymers such as:
polybutylenes such as INDOPOL H-100 (of molecular weight or MW=965 g/mol), INDOPOL H-300 (MW=1340 g/mol), INDOPOL H-1500 (MW=2160 g/mol) marketed or manufactured by the company AMOCO,
hydrogenated polyisobutylenes such as PANALANE H-300 E marketed or manufactured by the company AMOCO (MW=1340 g/mol), VISEAL 20000 marketed or manufactured by the company SYNTEAL (MW=6000 g/mol), REWOPAL PIB 1000 marketed or manufactured by the company WITCO (MW=1000 g/mol),
polydecenes and hydrogenated polydecenes such as: PURESYN 10 (MW=723 g/mol), PURESYN 150 (MW=9200 g/mol) marketed or manufactured by the company MOBIL CHEMICALS,
copolymers of vinylpyrrolidone such as: the vinylpyrrolidone/1-hexadecene copolymer, ANTARON V-216 marketed or manufactured by the company ISP (MW=7300 g/mol),
esters such as:
esters of linear fatty acids having a total number of carbons in the range from 35 to 70 such as pentaerythrityl tetrapelargonate (MW=697 g/mol),
hydroxylated esters such as polyglycerol-2 triisostearate (MW=965 g/mol),
aromatic esters such as tridecyl trimellitate (MW=757 g/mol),
esters of C₂₄-C₂₈ branched fatty alcohols or fatty acids such as those described in application EP-A-0 955 039, and notably triisoarachidyl citrate (MW=1033.76 g/mol), pentaerythrityl tetraisononanoate (MW=697 g/mol), glyceryl triisostearate (MW=891 g/mol), glyceryl tridecyl-2 tetradecanoate (MW=1143 g/mol), pentaerythrityl tetraisostearate (MW=1202 g/mol), polyglyceryl-2 tetraisostearate (MW=1232 g/mol) or pentaerythrityl tetradecyl-2 tetradecanoate (MW=1538 g/mol),
a polyester resulting from esterification of at least one triglyceride of hydroxylated carboxylic acid(s) by an aliphatic monocarboxylic acid and by an aliphatic dicarboxylic acid, optionally unsaturated such as castor oil of succinic acid and isostearic acid marketed under the reference Zenigloss by Zenitech,
esters of dimer diol and of dimer diacid of general formula HO—R¹—(—OCO—R²—COO—R¹—)ₕ—OH, in which:
   R¹ represents a dimer diol residue obtained by hydrogenation of dilinoleic diacid
   R² represents a hydrogenated dilinoleic diacid residue, and
   h represents an integer in the range from 1 to 9, notably the esters of dilinoleic diacids and of dilinoleic dimer diols marketed by the company NIPPON FINE CHEMICAL under the trade name LUSPLAN DD-DA5® and DD-DA7®,
silicone oils such as phenylated silicones (also called phenylated silicone oil) such as BELSIL PDM 1000 from the company WACKER (MW=9000 g/mol), phenyl trimethicones (such as the phenyl trimethicone sold under the trade name DC556 by Dow Corning), phenyl dimethicones, phenyl trimethylsiloxydiphenyl siloxanes, diphenyl dimethicones, diphenyl methyldiphenyl trisiloxanes;
oils of vegetable origin such as sesame oil (MW=820 g/mol),
and mixtures thereof.

The gloss oil can also be an oligomer of hydroxylated fatty acid triglyceride and of saturated diacid.

Said oligomer is obtained by reaction of a hydroxylated fatty acid triglyceride (such as hydrogenated castor oil) and of a saturated diacid.

According to the invention, the diacid is said to be saturated when the hydrocarbon chain of which it is constituted does not have an unsaturation, i.e. carbon-carbon double bond. Diacid means a hydrocarbon compound comprising two carboxyl functions —COOH. The diacid can be a single diacid or a mixture of several diacids.

Similarly, in the sense of the invention, the oligomer can be a mixture of several oligomers.

Among the saturated diacids that can be used, we may mention sebacic acid (or 1,10-decanedioic acid), succinic acid, adipic acid, azelaic acid, octadecamethylene dicarboxylic acid and eicosadicarboxylic acid.

More particularly, the oligomer can be an oligoester whose monomers are represented by the following formulae (A) of triglyceride and (B) of diacid:

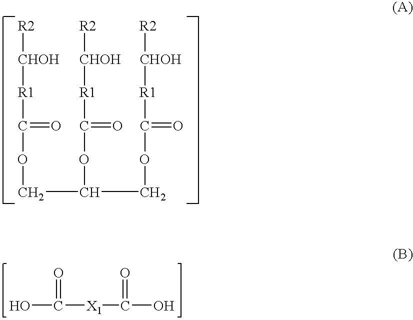

in which

R₁ represents a saturated or unsaturated, linear or branched alkylene group comprising for example from 1 to 18 carbon atoms, and R₂ represents a saturated or unsaturated, linear or branched alkyl group comprising for example from 1 to 12 carbon atoms;

R₁ preferably represents a group —(CH₂)ₙ—, where n can vary from 1 to 20 and notably from 3 to 16, for example from 6 to 12;

R₂ preferably represents a group —(CH₂)ₘCH₂, where m can vary from 0 to 11 and notably from 2 to 11, for example from 3 to 9;

According to one embodiment n=10 and m=5, and the group

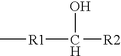

represents the alkyl residue of 12-hydroxystearic acid (the main component of hydrogenated castor oil);

X₁ is a linear or branched alkylene group, for example a linear alkylene group —(CH₂)ₓ—, where x can vary from 1 to 30 and notably from 3 to 15.

When the diacid is sebacic acid, x is equal to 8.

The average degree of polymerization of the oligomer can vary between 3 and 12.

The oligoester of hydrogenated castor oil and sebacic acid is notably marketed by the company CRODA under various names depending on the degree of polymerization.

Among the oligoesters formed from hydrogenated castor oil and sebacic acid, that having a degree of polymerization of about 4.6 is available under the trade name “CROMADOL CWS-5” and that having a degree of polymerization of about 9.5 is available under the trade name “CROMADOL CWS-10”, marketed by Croda Japan K.K.

We may also mention the oligomer of hydrogenated castor oil and sebacic acid sold under the name CRODABOND-CSA (MW=3500) by the company CRODA.

Preferably, the gloss oil has a refractive index greater than or equal to 1.45 and notably in the range from 1.45 to 1.6.

### 2. Volatile Oil:

The first and/or second fatty phases of the compositions of the product according to the invention can comprise at least one volatile oil.

“Volatile oil” means, in the sense of the invention, an oil that can evaporate when in contact with keratinous materials in less than an hour, at room temperature and atmospheric pressure (760 mmHg). The volatile organic solvent or solvents and the volatile oils of the invention are volatile organic solvents and cosmetic oils, liquid at room temperature, having a non-zero vapour pressure, at room temperature and atmospheric pressure, in particular in the range from 0.13 Pa to 40 000 Pa (10⁻³ to 300 mmHg), in particular in the range from 1.3 Pa to 13 000 Pa (0.01 to 100 mmHg), and more particularly in the range from 1.3 Pa to 1300 Pa (0.01 to 10 mmHg).

These oils can be hydrocarbon oils, silicone oils, fluorinated oils, or mixtures thereof.

a. Silicone Oil

According to a variant of the invention, the volatile oil is a volatile silicone oil.

“Silicone oil” means an oil comprising at least one silicon atom, and notably comprising Si—O groups.

The volatile silicone oil usable in the invention can be selected from the silicone oils having a flash point in the range from 40° C. to 102° C., preferably having a flash point above 55° C. and less than or equal to 95° C., and preferably in the range from 65° C. to 95° C.

The volatile silicone oil can be selected from the linear or cyclic silicone oils such as the linear or cyclic polydimethylsiloxanes (PDMS) having from 3 to 7 silicon atoms.

As examples of said oils, we may mention octyltrimethicone, hexyltrimethicone, decamethylcyclopentasiloxane (cyclopentasiloxane or D5), octamethylcyclotetrasiloxane (cyclotetradimethylsiloxane or D4), dodecamethylcyclohexasiloxane (D6), decamethyltetrasiloxane (L4), KF 96 A from Shin Etsu, polydimethysiloxanes such as those marketed under the reference DC 200 (1.5 cSt), DC 200 (5 cSt), DC 200 (3 cSt) by Dow Corning.

b. Hydrocarbon Oil

According to a variant of the invention, the volatile oil is a volatile hydrocarbon oil.

“Hydrocarbon oil” means an oil formed essentially, or even constituted, of carbon atoms and of hydrogen, and optionally of oxygen and nitrogen atoms, and not containing silicon or fluorine atoms. It can contain alcohol, ester, ether, carboxylic acid, amine and/or amide groups.

The volatile hydrocarbon oils (also called solvents) can be selected from the hydrocarbon oils having from 8 to 16 carbon atoms, and notably the C8-C16 branched alkanes such as the C8-C16 isoalkanes of petroleum origin (also called isoparaffins) such as isododecane (also called 2,2,4,4,6-pentamethylheptane), isodecane, isohexadecane, and for example the oils sold under the trade names Isopars' or Permethyls, the C8-C16 branched esters, isohexyl neopentanoate, and mixtures thereof. Other volatile hydrocarbon oils such as the petroleum distillates, notably those sold under the name Shell Solt by the company SHELL, can also be used. Preferably, the volatile solvent is selected from the volatile hydrocarbon oils having from 8 to 16 carbon atoms and mixtures thereof.

c. Fluorinated Oils

The volatile oil can also be selected from the fluorinated oils such as the perfluoropolyethers, the perfluoroalkanes such as perfluorodecalin, perfluorodamantanes, the monoesters, diesters and triesters of perfluoroalkylphosphates and fluorinated ester oils.

Advantageously, the second composition comprises at least one volatile oil.

Preferably, the volatile oil is a hydrocarbon oil.

Preferably, the first composition has a content of volatile oil of less than or equal to 50 wt. %, preferably less than or equal to 30 wt. % and more preferably less than or equal to 20 wt. % relative to the total weight of the composition.

Even more preferably, the first composition is free from volatile oil.

Preferably, the first composition has a content of volatile silicone oil less than or equal to 15 wt. %, preferably less than or equal to 10 wt. % and more preferably less than or equal to 5 wt. % relative to the total weight of the composition. Even more preferably, the first composition is free from volatile silicone oil.

Preferably, the second composition has a content of volatile silicone oil less than or equal to 15 wt. %, preferably less than or equal to 10 wt. % and more preferably less than or equal to 5 wt. % relative to the total weight of the composition. Even more preferably, the second composition is free from volatile silicone oil.

### 2. Solid Fats

Advantageously, the first and/or second fatty phase of the compositions of the product according to the invention can comprise at least one solid fat such as a pasty fat or a wax.

### Pasty Fats

“Pasty” means, in the sense of the present invention, a lipophilic fatty compound with reversible solid/liquid change of state, having an anisotropic crystalline organization in the solid state, and having a liquid fraction and a solid fraction at a temperature of 23° C.

In other words, the initial melting point of the pasty compound can be below 23° C. The liquid fraction of the pasty compound measured at 23° C. can represent 9 to 97 wt. % of the compound. This fraction that is liquid at 23° C. preferably represents between 15 and 85 wt. %, more preferably between 40 and 85 wt. %.

In the sense of the invention, the melting point corresponds to the temperature of the most endothermic peak observed in thermal analysis (DSC) as described in standard ISO 11357-3; 1999. The melting point of a pasty fat or of a wax can be measured by differential scanning calorimetry (DSC), for example using the calorimeter sold under the name “MDSC 2920” by the company TA Instruments.

The measurement protocol is as follows:

A 5 mg sample of pasty fat or of wax (as appropriate) in a crucible is submitted to a first temperature rise in the range from −20° C. to 100° C., at a heating rate of 10° C./min, then it is cooled from 100° C. to −20° C. at a cooling rate of 10° C./min and finally is submitted to a second temperature rise from −20° C. to 100° C. at a heating rate of 5° C./min. During the second temperature rise, the variation of the difference of power absorbed by the empty crucible and by the crucible containing the sample of wax or pasty fat is measured as a function of the temperature. The melting point of the compound is the value of the temperature corresponding to the top of the peak of the curve representing the variation of the difference in power absorbed as a function of temperature.

The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the compound in passing from the solid state to the liquid state. The pasty compound is said to be in the solid state when the whole of its mass is of crystalline solid form. The pasty compound is said to be in the liquid state when the whole of its mass is in liquid form.

The enthalpy of fusion of the pasty compound is equal to the area under the curve of the thermogram obtained using a differential scanning calorimeter (DSC), such as the calorimeter sold under the name MDSC 2920 by the company TA Instruments, with a temperature rise of 5 or 10° C. per minute, according to standard ISO 11357-3:1999. The enthalpy of fusion of the pasty compound is the amount of energy required to cause the compound to pass from the solid state to the liquid state. It is expressed in J/g.

The enthalpy of fusion consumed at 23° C. is the amount of energy absorbed by the sample in passing from the solid state to the state that it has at 23° C., constituted of a liquid fraction and a solid fraction.

The liquid fraction of the pasty compound measured at 32° C. preferably represents from 30 to 100 wt. % of the compound, preferably from 50 to 100 wt. %, more preferably from 60 to 100 wt. % of the compound. When the liquid fraction of the pasty compound measured at 32° C. is equal to 100%, the end point of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

The pasty compound is preferably selected from synthetic compounds and compounds of vegetable origin. A pasty compound can be obtained by synthesis from starting products of vegetable origin.

The pasty compound is advantageously selected from
lanolin and its derivatives
polyol ethers selected from the ethers of pentaerythritol and of polyalkylene glycol, ethers of fatty alcohol and of sugar, and mixtures thereof; ether of pentaerythritol and of polyethylene glycol having 5 ethoxylated units (5 EO) (CTFA name: PEG-5 Pentaerythrityl Ether), ether of pentaerythritol and of polypropylene glycol having 5 propoxylated units (5 PO) (CTFA name: PPG-5 Pentaerythrityl Ether), and mixtures thereof and more especially the mixture PEG-5 Pentaerythrityl Ether, PPG-5 Pentaerythrityl Ether and soya oil, marketed under the name “Lanolide” by the company Vevy, a mixture in which the constituents have the proportions by weight 46/46/8: 46% of PEG-5 Pentaerythrityl Ether, 46% of PPG-5 Pentaerythrityl Ether and 8% of soya oil.
silicone compounds, polymeric or non-polymeric
fluorinated compounds, polymeric or non-polymeric
vinylic polymers, notably:
   homopolymers of olefins (such as polyvinyl laurate)
copolymers of olefins
homopolymers and copolymers of hydrogenated dienes
linear or branched oligomers, homo or copolymeric, of alkyl (meth)acrylates preferably having a C₈-C₃₀ alkyl group
   homo and copolymeric oligomers of vinyl esters with C₈-C₃₀ alkyl groups
   homo and copolymeric oligomers of vinyl ethers with C₈-C₃₀ alkyl groups,
fat-soluble polyethers resulting from polyetherification between one or more C2-C100, preferably C2-C50, diols
esters,
and/or mixtures thereof.

The pasty compound is preferably a polymer, notably a hydrocarbon polymer.

Among the fat-soluble polyethers, the copolymers of ethylene oxide and/or of propylene oxide with C6-C30 long-chain alkylene oxides, more preferably such that the weight ratio of ethylene oxide and/or of propylene oxide to alkylene oxides in the copolymer is from 5:95 to 70:30, are particularly preferred. In this family, we may notably mention the copolymers such that the long-chain alkylene oxides are arranged in blocks having an average molecular weight from 1000 to 10 000, for example a polyoxyethylene/polydodecyl glycol block copolymer such as the ethers of dodecanediol (22 mol) and of polyethylene glycol (45 EO) marketed under the trade name ELFACOS ST9 by Akzo Nobel.

Among the esters, notably the following are preferred
esters of an oligomeric glycerol, notably the esters of diglycerol, in particular the condensates of adipic acid and of glycerol, for which some of the hydroxyl groups of the glycerols have reacted with a mixture of fatty acids such as stearic acid, capric acid, stearic acid and isostearic acid and 12-hydroxystearic acid, notably such as those marketed under the trade name Softisan 649 by the company Sasol
arachidyl propionate marketed under the trade name Waxenol 801 by Alzo,
phytosterol esters,
triglycerides of fatty acids and their derivatives
esters of pentaerythritol
non-crosslinked polyesters resulting from polycondensation between a dicarboxylic acid or a linear or branched C4-C50 polycarboxylic acid and a C2-C50 diol or polyol,
aliphatic esters of ester resulting from esterification of an aliphatic hydroxycarboxylic acid ester by an aliphatic carboxylic acid.

The aliphatic carboxylic acid comprises from 4 to 30 and preferably from 8 to 30 carbon atoms. It is preferably selected from hexanoic acid, heptanoic acid, octanoic acid, ethyl-2 hexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid, docosanoic acid, and mixtures thereof.

The aliphatic carboxylic acid is preferably branched. The ester of aliphatic hydroxycarboxylic acid is advantageously derived from a hydroxylated aliphatic carboxylic acid having from 2 to 40 carbon atoms, preferably from 10 to 34 carbon atoms and more preferably from 12 to 28 carbon atoms, and from 1 to 20 hydroxyl groups, preferably from 1 to 10 hydroxyl groups and more preferably from 1 to 6 hydroxyl groups. The ester of aliphatic hydroxycarboxylic acid is selected from:
a) the partial or total esters of linear, saturated monohydroxylated aliphatic monocarboxylic acids;
b) the partial or total esters of unsaturated monohydroxylated aliphatic monocarboxylic acids;
c) the partial or total esters of saturated monohydroxylated aliphatic polycarboxylic acids;
d) the partial or total esters of saturated polyhydroxylated aliphatic polycarboxylic acids;
e) the partial or total esters of C₂ to C₁₆ aliphatic polyols that have reacted with a mono- or poly-hydroxylated aliphatic mono- or poly-carboxylic acid, and mixtures thereof.

The aliphatic esters of ester are advantageously selected from:
the ester resulting from the reaction of esterification of hydrogenated castor oil with isostearic acid in the proportions 1 to 1 ( 1/1) or monoisostearate of hydrogenated castor oil,
the ester resulting from the reaction of esterification of hydrogenated castor oil with isostearic acid in the proportions 1 to 2 (½) or diisostearate of hydrogenated castor oil,
the ester resulting from the reaction of esterification of hydrogenated castor oil with isostearic acid in the proportions 1 to 3 (⅓) or triisostearate of hydrogenated castor oil,
and mixtures thereof.

According to a first embodiment, the pasty compound or compounds preferably represent(s) 0.1 to 80 wt. %, preferably 0.5 to 60 wt. %, more preferably 1 to 30 wt. % and even more preferably 1 to 20 wt. % of the first and/or of the second composition.

Preferably, when the first composition comprises a pasty fat, then the second composition also comprises one.

According to a second embodiment, the first and/or the second composition is free from pasty fats.

### Wax(es)

According to one embodiment of the invention, the first and/or the second composition comprises at least one wax.

The wax considered within the scope of the present invention is generally a lipophilic compound, solid at room temperature (25° C.), with reversible solid/liquid change of state, having a melting point greater than or equal to 30° C. which can be up to 200° C. and notably up to 120° C.

In particular, the waxes suitable for the invention can have a melting point greater than or equal to 45° C., and in particular greater than or equal to 55° C.

The waxes that can be used in the compositions according to the invention are selected from waxes, solid at room temperature, of animal, vegetable, mineral or synthetic origin and mixtures thereof.

By way of illustration of waxes that are suitable for the invention, we may notably mention the hydrocarbon waxes such as beeswax, lanolin wax, and Chinese insect waxes, rice bran wax, carnauba wax, candelilla wax, ouricury wax, alfa wax, berry wax, shellac wax, Japan wax and sumac wax; montan wax, orange and lemon waxes, microcrystalline waxes, paraffins and ozokerite; polyethylene waxes, waxes obtained by Fischer-Tropsch synthesis and waxy copolymers as well as their esters.

We may also mention the waxes obtained by catalytic hydrogenation of animal or vegetable oils having linear or branched, C₈-C₃₂ fatty chains. Among the latter, we may notably mention isomerized jojoba oil such as the partially hydrogenated transisomerized jojoba oil manufactured or marketed by the company DESERT WHALE under the trade reference Iso-Jojoba-50®, hydrogenated sunflower oil, hydrogenated castor oil, hydrogenated copra oil, hydrogenated lanolin oil, and di(trimethylol-1,1,1-propane) tetrastearate sold under the name Hest 2T-4S® by the company HETERENE.

We may also mention the silicone waxes (C₃₀₋₄₅ ALKYL DIMETHICONE), and fluorinated waxes.

It is also possible to use the waxes obtained by hydrogenation of castor oil esterified with cetyl alcohol sold under the names Phytowax ricin 16L64® and 22L73® by the company SOPHIM. Said waxes are described in application FR-A-2792190.

It is possible to use, as wax, a C₂₀-C₄₀ alkyl (hydroxystearyloxy)stearate (the alkyl group comprising from 20 to 40 carbon atoms), alone or mixed.

Such a wax is notably sold under the names “Kester Wax K 82 P®”, “Hydroxypolyester K 82 P®” and “Kester Wax K 80 P®” by the company KOSTER KEUNEN.

As microwaxes that can be used in the compositions according to the invention, we may notably mention the carnauba microwaxes such as that marketed under the name MicroCare 350® by the company MICRO POWDERS, the microwaxes of synthetic wax such as that marketed under the name MicroEase 114S® by the company MICRO POWDERS, the microwaxes constituted of a mixture of carnauba wax and of polyethylene wax such as those marketed under the names MicroCare 300® and 310® by the company MICRO POWDERS, the microwaxes constituted of a mixture of carnauba wax and of synthetic wax such as that marketed under the name MicroCare 325® by the company MICRO POWDERS, the polyethylene microwaxes such as those marketed under the names Micropoly 200®, 220®, 220L® and 250S® by the company MICRO POWDERS and the polytetrafluoroethylene microwaxes such as those marketed under the names Microslip 519® and 519 L® by the company MICRO POWDERS.

According to a first embodiment, the first and/or the second composition of the product according to the invention can have a content of waxes in the range from 0.1 to 30 wt. % relative to the total weight of the composition, in particular its content thereof can be from 0.5 to 20%, more particularly from 1 to 15%.

Preferably, when one of the compositions of the product according to the invention comprises a wax, then the other also comprises one, and preferably the waxes of the different compositions are of the same nature, for example hydrocarbon waxes.

According to another embodiment, the first and/or the second composition according to the invention is free from wax.

### Physiologically Acceptable Medium

The first and the second composition of the product of the invention must be cosmetically or dermatologically acceptable, i.e. must contain a physiologically acceptable, non-toxic medium that can be applied on the lips of human beings. “Cosmetically acceptable” means, in the sense of the invention, a composition of pleasant appearance, odour and feel.

### Structurizing Agent/Thickener

The first and/or the second composition of the product according to the invention can comprise, in addition to the waxes that are optionally present, a structurizing agent selected from semi-crystalline polymers, lipophilic gelling agents and mixtures thereof.

### Semi-Crystalline Polymers

“Semi-crystalline polymer” means compounds having at least two repeating units, preferably at least 3 repeating units and more especially at least 10 repeating units. “Semi-crystalline polymer” means polymers having a crystallizable part, a crystallizable pendant chain or a crystallizable block in the backbone, and an amorphous part in the backbone and having a reversible first-order phase transition temperature, in particular melting point (solid-liquid transition). When the crystallizable part is in the form of a crystallizable block of the polymer backbone, the amorphous part of the polymer is in the form of an amorphous block; the semi-crystalline polymer is in this case a block copolymer for example of the diblock, triblock or multiblock type, having at least one crystallizable block and at least one amorphous block. “Block” generally means at least 5 identical repeating units. The crystallizable chain or blocks is/are then of different chemical nature from the amorphous chain or blocks.

The semi-crystalline polymer has a melting point greater than or equal to 30° C. (notably in the range from 30° C. to 80° C.), preferably in the range from 30° C. to 60° C. This melting point is a temperature of a first-order change of state.

This melting point can be measured by any known method and in particular by means of a differential scanning calorimeter (DSC).

Advantageously, the semi-crystalline polymer or polymers to which the invention applies have a number-average molecular weight greater than or equal to 1000. Advantageously, the semi-crystalline polymer or polymers of the composition of the invention have a number-average molecular weight □Mn in the range from 2000 to 800 000, preferably from 3000 to 500 000, more preferably from 4000 to 150 000, and notably less than 100 000, and preferably from 4000 to 99 000. Preferably, they have a number-average molecular weight greater than 5600, for example in the range from 5700 to 99 000. “Crystallizable chain or block” means, in the sense of the invention, a chain or block which, if alone, would pass from the amorphous state to the crystalline state, reversibly, depending on whether the temperature is above or below the melting point. A chain in the sense of the invention is a group of atoms that is pendant or lateral relative to the backbone of the polymer. A block is a group of atoms belonging to the backbone, said group constituting one of the repeating units of the polymer. Advantageously, the “crystallizable pendant chain” can be a chain having at least 6 carbon atoms.

The semi-crystalline polymer can be selected from the block copolymers having at least one crystallizable block and at least one amorphous block, the homopolymers and the copolymers having at least one crystallizable side chain per repeating unit, and mixtures thereof.

Such polymers are described for example in document EP 1396259.

According to a more particular embodiment of the invention, the polymer is derived from a monomer with a crystallizable chain selected from the saturated C₁₄ to C₂₂ alkyl (meth)acrylates.

As a particular example of structurizing semi-crystalline polymers for use in the composition according to the invention, we may mention the Intelimer® products from the company Landec described in the brochure “Intelimer® polymers”, Landec IP22 (Rev. 4-97). These polymers are in solid form at room temperature (25° C.). They bear crystallizable side chains and have the formula X given above.

### Lipophilic Gelling Agents

The gelling agents to be used in the compositions according to the invention can be organic or mineral, polymeric or molecular lipophilic gelling agents.

As mineral lipophilic gelling agent, we may mention the clays, optionally modified, such as hectorites modified with a C₁₀ to C₂₂ ammonium chloride, such as hectorite modified with distearyl dimethyl ammonium chloride, for example that marketed under the name Bentone 38V® by the company ELEMENTIS.

We may also mention pyrogenated silica optionally with hydrophobic surface treatment, with particle size less than 1 μm. It is in fact possible for the surface of silica to be modified chemically, by a chemical reaction producing a decrease in the number of silanol groups present on the surface of the silica. It is notably possible to replace the silanol groups with hydrophobic groups: a hydrophobic silica is then obtained. The hydrophobic groups can be:
trimethylsiloxyl groups, which are notably obtained by treatment of pyrogenated silica in the presence of hexamethyldisilazane. Silicas thus treated are called “Silica silylate” according to the CTFA (8th edition, 2000). They are for example marketed under the references Aerosil R812® by the company DEGUSSA, CAB-O-SIL TS-530® by the company CABOT,
dimethylsilyloxyl or polydimethylsiloxane groups, which are notably obtained by treatment of pyrogenated silica in the presence of polydimethylsiloxane or of dimethyldichlorosilane. Silicas thus treated are called “Silica dimethyl silylate” according to the CTFA (8th edition, 2000). They are for example marketed under the references Aerosil R972®, and Aerosil R974® by the company DEGUSSA, CAB-O-SIL TS-610® and CAB-O-SIL TS-720® by the company CABOT.

Hydrophobic pyrogenated silica has in particular a particle size from nanometric to micrometric, for example in the range from about 5 to 200 nm.

The organic polymeric lipophilic gelling agents are for example partially or fully crosslinked elastomeric organopolysiloxanes, of three-dimensional structure, such as those marketed under the names KSG6®, KSG16® and of KSG18® by the company SHIN-ETSU, Trefil E-505C® and Trefil E-506C® by the company DOW-CORNING, Gransil SR-CYC®, SR DMF10®, SR-DC556®, SR 5CYC Gel®, SR DMF 10 Gel® and SR DC 556 Gel® by the company GRANT INDUSTRIES, SF 1204® and JK 113® by the company GENERAL ELECTRIC; ethylcellulose such as that sold under the name Ethocel® by the company DOW CHEMICAL; galactomannans having from one to six, and in particular from two to four, hydroxyl groups per monosaccharide unit, substituted with a saturated or unsaturated alkyl chain, such as guar gum alkylated by C₁ to C₆, and in particular C₁ to C₃, alkyl chains, and mixtures thereof. The block copolymers of the “diblock”, “triblock” or “radial” type of the polystyrene/polyisoprene, polystyrene/polybutadiene type such as those marketed under the name Luvitol HSB® by the company BASF, of the polystyrene/copoly(ethylene-propylene) type such as those marketed under the name Kraton® by the company SHELL CHEMICAL CO or of the polystyrene/copoly(ethylene-butylene) type, mixtures of triblock and radial (star) copolymers in isododecane such as those marketed by the company PENRECO under the name Versagel® for example the mixture of butylene/ethylene/styrene triblock copolymer and of ethylene/propylene/styrene star copolymer in isododecane (Versagel M 5960).

As lipophilic gelling agents, we may also mention the polymers of weight-average molecular weight below 100 000, having a) a polymer backbone with hydrocarbon repeating units provided with at least one heteroatom, and optionally b) at least one pendant fatty chain and/or at least one terminal fatty chain, optionally functionalized, having from 6 to 120 carbon atoms and being bound to said hydrocarbon units, as described in applications WO-A-02/056847, WO-A-02/47619 the contents of which are incorporated as reference; in particular the polyamide resins (notably comprising alkyl groups having from 12 to 22 carbon atoms) such as those described in U.S. Pat. No. 5,783,657, the contents of which are incorporated as reference.

Among the lipophilic gelling agents that can be used in the compositions according to the invention, we may also mention the esters of dextrin and of fatty acid, such as the dextrin palmitates, notably such as those marketed under the names Rheopearl TL® or Rheopearl KL® by the company CHIBA FLOUR.

It is also possible to use the silicone polyamides of the polyorganosiloxane type such as those described in documents U.S. Pat. No. 5,874,069, U.S. Pat. No. 5,919,441, U.S. Pat. No. 6,051,216 and U.S. Pat. No. 5,981,680.

These silicone polymers can belong to the following two families:
polyorganosiloxanes having at least two groups capable of establishing hydrogen interactions, these two groups being located in the polymer chain, and/or
polyorganosiloxanes having at least two groups capable of establishing hydrogen interactions, these two groups being located on grafts or branchings.

### Active Ingredients

The first composition and/or the second composition of the product according to the invention can comprise at least one “active ingredient” different from the polyhydric alcohols.

“Active ingredient” means a compound having a cosmetic and/or dermatological effect notably on the lips.

This active ingredient can be hydrophilic or hydrophobic. The active ingredient can be water-soluble.

Thus, the active ingredient present in the composition according to the invention can be selected independently from:
dermorelaxants,
agents stimulating the synthesis of dermal or epidermal macromolecules and/or preventing their
degradation,
anti-glycation agents,
anti-irritant agents,
hydrating agents other than polyhydric alcohols,
desquamating agents
pigmentation modifying agents,
NO-synthase inhibitors,
agents stimulating the proliferation of fibroblasts or of keratinocytes and/or the differentiation of keratinocytes,
anti-pollution or anti-radical agents,
soothing agents,
agents acting on the microcirculation,
agents acting on the energy metabolism of the cells,
cicatrizing agents, and
mixtures thereof.

The amount of active ingredient(s) is in the range for example from 0.0001 to 30 wt. % and preferably from 0.01 to 10 wt. % of active substance relative to the total weight of the composition.

Film-Forming Agent

The first and/or the second composition of the product according to the invention can comprise at least one film-forming polymer.

“Polymer” means, in the sense of the invention, a compound having at least 2 repeating units, and preferably at least 3 repeating units.

“Film-forming polymer” means a polymer that is able to form, by itself or in the presence of an auxiliary film-forming agent, a macroscopically continuous film on a substrate, notably on keratinous materials.

The polymer can be present in the first and/or in the second composition at a content in the range from 0.1 to 60 wt. %, relative to the total weight of the composition, preferably in the range from 0.1 to 50 wt. %, preferably in the range from 0.5 to 40 wt. %, preferably in the range from 1 to 30 wt. %, and more preferably in the range from 1 to 25 wt.%.

In one embodiment, the film-forming organic polymer is at least one polymer selected from the group comprising:
film-forming polymers soluble in an organic liquid medium, in particular fat-soluble polymers, when the organic liquid medium comprises at least one oil;
film-forming polymers dispersible in an organic solvent medium, in particular polymers in the form of non-aqueous dispersions of particles of polymers, preferably dispersions in silicone or hydrocarbon oils; in one embodiment, the non-aqueous dispersions of polymer comprise particles of polymers stabilized on their surface by at least one stabilizing agent;
film-forming polymers in the form of aqueous dispersions of polymer particles, often called “latex”; in this case, the composition comprises an aqueous phase;
water-soluble film-forming polymers; in this case, the composition comprises an aqueous phase.

Among the film-forming polymers for use in the composition of the present invention, we may mention synthetic polymers, of the radical type or of the polycondensate type, polymers of natural origin and mixtures thereof. As film-forming polymer, we may mention in particular acrylic polymers, polyurethanes, polyesters, polyamides, polyureas, silicone polymers, silicone grafted acrylic polymers, polyamide polymers and copolymers, polyisoprenes.

The composition according to the invention can comprise a plasticizer that promotes the formation of a film with the film-forming polymer. Said plasticizer can be selected from all the compounds that are known by a person skilled in the art to be able to perform the desired function.

### Silicone Resins

The first and/or the second composition of the product according to the invention can comprise at least one silicone resin.

As examples of silicone resins, we may mention:
siloxysilicates, which can be trimethylsiloxysilicates of formula [(CH₃)₃SiO]ₓ(SiO_{4/2})_{y} (MQ units) in which x and y are integers in the range from 50 to 80,
polysilesquioxanes of formula (CH₃SiO_{3/2}).ₓ (T units) in which x is greater than 100 and in which at least one of the methyl radicals can be substituted with a group R as defined above,
polymethylsilsesquioxanes, which are polysilsesquioxanes in which none of the methyl radicals is substituted with another group. Said polymethylsilsesquioxanes are described in document U.S. Pat. No. 5,246,694, the contents of which are incorporated by reference.

As examples of commercially available polymethylsilsesquioxane resins, we may mention those that are marketed:
by the company Wacker under the reference Resin MK such as Belsil PMS MK: polymer comprising CH₃SiO_{3/2} repeating units (T units), which can also comprise up to 1 wt. % of (CH₃)₂SiO_{2/2} units (D units) and having an average molecular weight of about 10 000,
by the company SHIN-ETSU under the references KR-220L which are composed of T units of formula CH₃SiO_{3/2} and have Si—OH (silanol) end groups, under the reference KR-242A which comprise 98% of T units and 2% of dimethyl units D and have Si—OH end groups or under the reference KR-251 comprising 88% of T units and 12% of dimethyl units D and have Si—OH end groups.

As siloxysilicate resins, we may mention the trimethylsiloxysilicate (TMS) resins optionally in the form of powders. These resins are marketed under the reference SR1000 by the company General Electric or under the reference TMS 803 by the company Wacker. We may also mention the trimethylsiloxysilicate resins marketed in a solvent such as cyclomethicone, sold under the name “KF-7312J” by the company Shin-Etsu, “DC 749”, “DC 593” by the company Dow Corning.

Advantageously, the silicone resin, such as the trimethylsiloxysilicate resin for example, is present at a content in the range from 0.5 to 30%, or preferably from 1 to 25% or better still from 5 to 25%.

### Fillers

The first and/or the second composition of the product according to the invention can comprise at least one filler, notably at a content in the range from 0.01 to 50 wt. %, relative to the total weight of the composition, preferably in the range from 0.01 to 30 wt. %. “Fillers” are to be understood as particles of any shape, colourless or white, mineral or synthetic, insoluble in the medium of the composition regardless of the temperature at which the composition is manufactured. These fillers notably serve for modifying the rheology or the texture of the composition.

The fillers can be mineral or organic of any shape, plate-shaped, spherical or oblong, regardless of the crystallographic form (for example leaf, cubic, hexagonal, orthorhombic, etc). We may mention talc, mica, silica, kaolin, powders of polyamide)(Nylon®) (Orgasol® from Atochem), of poly-β-alanine and of polyethylene, powders of tetrafluoroethylene polymers (Teflon®), lauroyl lysine, starch, boron nitride, hollow polymer microspheres such as those of polyvinylidene/acrylonitrile chloride such as Expancel® (Nobel Industry), of acrylic acid copolymers (Polytrap® from the company Dow Corning) and microbeads of silicone resin (Tospearls® from Toshiba, for example), particles of elastomeric polyorganosiloxanes, precipitated calcium carbonate, magnesium carbonate and hydrocarbonate, hydroxyapatite, hollow silica microspheres (Silica Beads® from Maprecos), glass or ceramic microcapsules, metal soaps derived from organic carboxylic acids having from 8 to 22 carbon atoms, preferably from 12 to 18 carbon atoms, for example zinc, magnesium or lithium stearate, zinc laurate, magnesium myristate.

### Usual Cosmetic Additives

The composition according to the invention can additionally comprise any usual cosmetic ingredient, which can notably be selected from antioxidants, perfumes, preservatives, neutralizing agents, surfactants, sun filters, vitamins, self-tanning compounds, antiwrinkle agents, emollients, anti-free-radical agents, deodorants, sequestering agents, film-forming agents, and mixtures thereof.

Of course, a person skilled in the art will take care to select optional supplementary ingredients and/or their amount in such a way that the advantageous properties of the composition according to the invention are not, or are substantially not, altered by the addition envisaged.

Of course, the amount of these supplementary compounds can be adjusted by a person skilled in the art so as not to be detrimental to the effect that is desired within the scope of the present invention.

### Non-Transfer

The first composition and/or the second composition can also be selected so that the product, once applied on keratinous materials, has satisfactory properties of non-transfer.

In particular, the first composition preferably has a transfer index below 35 and preferably less than or equal to 30, preferably less than or equal to 20, preferably less than or equal to 15, preferably less than or equal to 10, preferably less than or equal to 5, and more preferably less than or equal to 2 percent.

The second composition can be selected in such a way that the transfer index of the product of the invention is less than or equal to 35, preferably less than or equal to 30, preferably less than or equal to 20, preferably less than or equal to 15, preferably less than or equal to 10, and preferably less than or equal to 5.

The transfer index can be measured by the following method.

A substrate (rectangle of 40 mm×70 mm and with thickness of 3 mm) of polyethylene foam with adhesive on one of the faces having a density of 33 kg/m³ (sold under the name RE40X70EP3 by the company JOINT TECHNIQUE LYONNAIS IND) is preheated on a heating plate maintained at a temperature of 40° C. so that the surface of the substrate is maintained at a temperature of 33° C.±1° C.

With the substrate still on the heating plate, the composition is applied on all of the non-adhesive surface of the substrate, spreading it by means of a brush to obtain a deposit of the composition of about 15 μm, then it is left to dry for 30 minutes.

After drying, the substrate is glued by its adhesive face on an anvil with a diameter of 20 mm that is provided with a screw thread. The substrate/deposit assembly is then cut off using a punch with a diameter of 18 mm. The anvil is then screwed onto a press (STATIF MANUEL IMADA SV-2 from the company SOMECO) equipped with a dynamometer (IMADA DPS-20 from the company SOMECO).

A sheet of white photocopier paper of 80 g/m² is placed on the base of the press and then the substrate/deposit assembly is pressed onto the paper at a pressure of 2.5 kg for 30 seconds. After withdrawing the substrate/deposit assembly, some of the deposit has transferred to the paper. Then the colour of the deposit transferred onto the paper is measured using a MINOLTA CR300 colorimeter, the colour being characterized by the colorimetric parameters L*, a*, b*. The colorimetric parameters L*₀, a*₀, b*₀ of the colour of the blank paper used are determined.

Then the colour difference ΔE1 between the colour of the deposit transferred relative to the colour of the blank paper is determined from the following relation.

ΔE1=\sqrt{(L*−Lₒ*)²+(a*−aₒ*)²+(b*−bₒ*)²}

In addition, a total transfer reference is prepared by applying the composition directly on paper identical to that used previously, at room temperature (25° C.), spreading the composition by means of a brush so as to obtain a deposit of the composition of about 15 μm and then leaving it to dry for 30 minutes at room temperature (25° C.). After drying, the colorimetric parameters L*′, a*′, b*′ of the colour of the deposit put on the paper, corresponding to the total transfer reference colour, are measured directly. The colorimetric parameters L*′₀, a*′₀, b*′₀ of the colour of the blank paper used are determined.

Then the colour difference ΔE2 between the total transfer reference colour relative to the colour of the blank paper is determined from the following relation.

ΔE2=\sqrt{(L*′−Lₒ*′)²+(a*′−aₒ*′)²+(b*′−bₒ*′)²}

The transfer of the composition, expressed as a percentage, is equal to the ratio:

100×ΔE1/ΔE2

Measurement is carried out on 4 substrates in succession and the transfer value corresponds to the mean value of the 4 measurements obtained with the 4 substrates.

### Durability of Colour

The first composition and/or the second composition can also be selected so that the product, once applied on keratinous materials, has satisfactory properties of durability.

In particular, the first composition preferably has a durability index greater than or equal to 80%, preferably greater than or equal to 85%, preferably less than or equal to 90%, preferably greater than or equal to 95%.

The second composition can be selected in such a way that the durability index of the product is greater than or equal to 80%, preferably greater than or equal to 85%, preferably less than or equal to 90%, preferably greater than or equal to 95%.

The durability index of the deposit obtained with the composition according to the invention is determined according to the measurement protocol described below.

A substrate is prepared (rectangle of 40 mm×70 mm) constituted of an acrylic coating (hypoallergenic acrylic adhesive on polyethylene film sold under the name BLENDERME ref FH5000-55113 by the company 3M Santé) glued on a layer of polyethylene foam adhesive on the face opposite to that on which the sticking plaster is fixed (layer of foam sold under the name RE40X70EP3 by the company JOINT TECHNIQUE LYONNAIS IND).

The L*₀a*₀b*₀ colour of the substrate, on the face with the acrylic coating, is measured using a MINOLTA CR 300 colorimeter.

The substrate prepared in this way is preheated on a heating plate maintained at a temperature of 40° C. so that the surface of the substrate is maintained at a temperature of 33° C.±1° C.

With the substrate still on the heating plate, the composition is applied on all of the non-adhesive surface of the substrate (i.e. on the surface of the acrylic coating) spreading it by means of a brush to obtain a deposit of the composition of about 15 μm and then it is left to dry for 10 minutes.

After drying, the L*a*b* colour of the film thus obtained is measured.

Then the colour difference ΔE1 between the colour of the film relative to the colour of the bare substrate is determined from the following relation.

ΔE1=\sqrt{(L*−Lₒ*)²+(a*−aₒ*)²+(b*−bₒ*)²}

The substrate is then glued by its adhesive face (adhesive face of the layer of foam) on an anvil with a diameter of 20 mm that is provided with a screw thread. A test specimen of the substrate/deposit assembly is then cut out by means of a punch with a diameter of 18 mm. The anvil is then screwed onto a press (STATIF MANUEL IMADA SV-2 from the company SOMECO) equipped with a dynamometer (IMADA DPS-20 from the company SOMECO).

On white photocopier paper with a weight of 80 g/m², a strip 33 mm wide and 29.7 cm long is drawn, making a first line at 2 cm from the edge of the sheet, then a second line at 5 cm from the edge of the sheet, the first and second lines thus delimiting a box on the strip; then a first mark and a second mark are arranged, located in the strip respectively at the 8 cm and 16 cm points on the second line. On the first mark we put 20 μl of water and on the second mark 10 μl of refined sunflower oil (sold by the company LESIEUR).

The white paper is placed on the base of the press and then the test specimen, placed on the box of the strip of paper, is pressed at a pressure of about 300 g/cm² applied for 30 seconds. Then the press is raised and the test specimen is again placed just after the second line (therefore alongside the box), a pressure of about 300 g/cm² is applied again and the paper is moved, rectilinearly once contact is made, at a speed of 1 cm/s, over the full length of the strip in such a way that the test specimen passes through the deposits of water and oil.

After withdrawing the test specimen, some of the deposit has transferred onto the paper. The L*′, a*′, b*′ colour of the deposit that remains on the test specimen is then measured.

Then the colour difference ΔE2 between the colour of the deposit left behind on the test specimen relative to the colour of the bare substrate is determined from the following relation.

ΔE2=\sqrt{(L*′−Lₒ*)²+(a*′−aₒ*)²+(b*′−bₒ*)²}

The durability index of the composition, expressed as a percentage, is equal to the ratio:

100×ΔE2/ΔE1

Measurement is effected on 6 substrates in succession and the transfer value corresponds to the mean value of the 6 measurements obtained with the 6 substrates.

The first composition can contain, in addition to the silicone polyamide and the tackifying resin, at least one oil, another silicone polymer, a surfactant, a colouring agent, a filler, an active ingredient, and a mixture thereof.

The compositions of the product can be in the form of a cast product and for example in the form of a stick, or in the form of a dish for use by direct contact or with a sponge. In particular, they find application as cast foundation, cast blusher or eye shadow, lipstick, or concealer. They can also be in the form of a soft paste or a gel, a more or less fluid cream, or liquid packaged in a tube.

Cosmetically acceptable means, in the sense of the invention, a composition with a pleasant appearance, odour and feel.

Each composition of the product according to the invention can be in any dosage form normally used for topical application and notably in the form of a solution in oil or water, or an oily gel.

Preferably, the first and second compositions are independently oily solutions in the form of liquid, solid or a soft paste of intermediate texture.

According to one method of application, the first or the second composition, or both, have an oily continuous phase.

Each first and second composition can have the form of a lotion, a cream, an ointment, a soft paste, an unguent, a cast or moulded solid and notably as a stick or in a dish.

Each composition can be packaged separately in one and the same packaging article for example in a two-compartment pen, the base composition being delivered by one end of the pen and the top composition being delivered by the other end of the pen, each end being closed notably hermetically with a cap.

The product according to the invention can be used advantageously for making up the skin and/or the lips and/or the integumentary appendages depending on the nature of the ingredients used. In particular, the product of the invention can be in the form of a solid foundation, lipstick in the form of a stick or as paste, concealer, or for the contours of the eyes, eye liner, eye shadow, or a product for making up the body.

The invention further relates to a product for the lips, a foundation, a blusher or eye shadow comprising a first composition and a second composition as described previously.

Preferably, the product according to the invention is a lipstick.

The compositions of the product of the invention can be obtained by heating the different constituents to a temperature corresponding to the highest melting point of the ingredient or ingredients, then casting the molten mixture in a mould (dish or finger-shaped mould). They can also be obtained by extrusion as described in application EP-A-0 667 146.

The invention is illustrated in more detail in the following examples. The percentages are percentages by weight.

### EXAMPLES

### Lipsticks

For greater clarity, the compositions comprising at least one hydrating agent and corresponding to the first compositions of the products according to the invention are designated “A” and the second compositions of the products according to the invention are called compositions “B”.

### Examples 1 and 2

### First Compositions A1 and A2 (each Comprising a Hydrating Agent)

Compositions A1 (solid) and A2 (liquid) in the examples are obtained according to the following protocol.

Firstly, the fillers and the pigments are ground in a portion of the oily phase.

The remaining fat-soluble ingredients are then mixed at a temperature of the order of 100° C. The ground product or the pre-dispersed active ingredients are then added to the oily phase.

### Example A1

### Solid Formula Comprising Glycerol as Hydrating Agent

| Composition A1 | Type | Concentration |
|---|---|---|
| ISONONYL ISONONANOATE | FAT | q.s. |
| PENTAERYTHRITYL TETRAISOSTEARATE | FAT | 12 |
| GLYCERYL ESTERS OF VEGETABLE | FAT | 16 |
| ISOSTEARIC, ADIPIC FATTY ACIDS | | |
| OLEYL ERUCATE | FAT | 15 |
| MICROCRYSTALLINE WAX (C20-C60) | FAT | 7.5 |
| CARNAUBA WAX | FAT | 2.3 |
| RUTILE TITANIUM DIOXIDE TREATED | COLORANT | 1.35 |
| WITH | | |
| ALUMINA/SILICA/TRIMETHYOLPROPANE | | |
| (CI: 77891) | | |
| ALUMINIUM LAKE OF BRILLANT YELLOW | COLORANT | 0.45 |
| FCF ON ALUMINA (42/58) (CI: | | |
| 15985:1 + 77002) | | |
| BLACK IRON OXIDE (CI: 77499) | COLORANT | 0.2 |
| ALUMINIUM LAKE OF DISODIUM SALT OF | COLORANT | 0.23 |
| PHLOXINE B ON ALUMINA, ALUMINIUM | | |
| BENZOATE | | |
| MICA-TITANIUM DIOXIDE (56/44) | NACRE | 8 |
| POLYVINYL LAURATE (Mexomere PP | POLYMER | 4 |
| from Chimex) | | |
| TRIOLEYL PHOSPHATE | SURFACTANT | 1 |
| GLYCEROL | HYDRATING | 8 |
| | AGENT | |
| PYROGENATED SILICA OF HYDROPHOBIC | FILLER | 2.4 |
| CHARACTER (AEROSIL R 972 from EVONIK DEGUSSA) | | |
| Total: | | 100 |

“q.s.” means, in the various formulas, the complement by weight so that the total of the percentages by weight of the various compounds is equal to 100.

### Example A2

### Liquid Formula Comprising Glycerol

| Composition A2 | Type | Concentration |
|---|---|---|
| TRIGLYCERIDE OF 2-DECYL | FAT | 1 |
| TETRADECANOIC ACID (GUERBET C24) | | |
| GLYCERYL TRIHEPTANOATE | FAT | 4 |
| PENTAERYTHRITYL TETRAISOSTEARATE | FAT | 7.5 |
| DIISOSTEARYL MALATE | FAT | q.s. |
| BEESWAX | FAT | 7 |
| UNPROTECTED PERHYDROSQUALENE | FAT | 5 |
| OCTYL-2 DODECANOL | FAT | 10 |
| RUTILE TITANIUM DIOXIDE TREATED | COLORANT | 0.11 |
| WITH | | |
| ALUMINA/SILICA/TRIMETHYOLPROPANE | | |
| (CI: 77891) | | |
| ALUMINIUM LAKE OF BRILLIANT YELLOW | COLORANT | 0.01 |
| FCF ON ALUMINA (42/58) (CI: | | |
| 15985:1 + 77002) | | |
| BLACK IRON OXIDE (CI: 77499) | COLORANT | 0.001 |
| ALUMINIUM LAKE OF DISODIUM SALT OF | COLORANT | 0.01 |
| PHLOXINE B ON ALUMINA, ALUMINIUM | | |
| BENZOATE (CI: 45410:2 + 77002) | | |
| POLYGLYCEROL TRIISOSTEARATE (2 MOL) | SURFACTANT | 11 |
| TRIOLEYL PHOSPHATE | SURFACTANT | 1 |
| GLYCEROL | HYDRATING | 7.5 |
| | AGENT | |
| HYDROPHOBIC PYROGENATED SILICA, | FILLER | 7.5 |
| SURFACE TREATED WITH | | |
| DIMETHYLSILANE (AEROSIL R 972 from | | |
| EVONIK DEGUSSA) | | |
| Total: | | 100 |

### Examples 3 to 9

### Examples of Second Compositions Comprising at Least One Nonvolatile Ester Oil and Free from Hydrating Agent

The second compositions B3, B4, B5, B6, B7, B8 and B9 of examples 3 to 6 respectively are obtained according to the following protocol.

Firstly, the fillers and the pigments are ground in a portion of the oily phase.

The rest of the fat-soluble ingredients are then mixed at a temperature of the order of 100° C.

### Example B3

### Solid Formula Comprising at Least One Nonvolatile Ester Oil and Free from Hydrating Agent

| | | Percentage |
|---|---|---|
| Composition B3 | Type | by weight |
| ISONONYL ISONONANOATE | FAT | 8.5 |
| TRIGLYCERIDE OF 2-DECYL | FAT | 3 |
| TETRADECANOIC ACID (GUERBET C24) | | |
| TRIDECYL TRIMELLITATE | FAT | q.s. |
| PENTAERYTHRITYL TETRAISOSTEARATE | FAT | 14.5 |
| DIISOSTEARYL MALATE | FAT | 14.5 |
| GLYCERYL ESTERS OF VEGETABLE | FAT | 7 |
| ISOSTEARIC, ADIPIC FATTY ACIDS | | |
| OCTYL-2-DODECYL STEARATE | FAT | 5 |
| MICROCRYSTALLINE WAX (C20-C60) | FAT | 4 |
| RED IRON OXIDE (CI: 77491) | COLORANT | 3.3 |
| BLACK IRON OXIDE (CI: 77499) | COLORANT | 0.01 |
| CALCIUM SALT OF RED LITHOL B | COLORANT | 0.12 |
| ALUMINIUM LAKE OF BRILLIANT YELLOW | COLORANT | 0.23 |
| FCF ON ALUMINA (42/58) (CI: 15985:1 + 77002) | | |
| MICA-TITANIUM DIOXIDE (65/35) | NACRE | 2 |
| MICA-TITANIUM DIOXIDE (67.5/32.5) | NACRE | 3 |
| POLYETHYLENE WAX (MW: 500) | POLYMER | 6.3 |
| POLYBUTYLENE (MONOOLEFINS/ | POLYMER | 5 |
| ISOPARAFFINS 95/5) (MW: 2060) | | |
| MIXTURE OF SOYA STEROLS, | SURFACTANT | 9 |
| ETHOXYLATED (5 EO) PROPOXYLATED (5 PO) PENTAERYTHRITOL | | |
| Total: | | 100 |

### Example B4

### Solid Formula Comprising at Least One Nonvolatile Ester Oil and Free from Hydrating Agent

| | | Percentage |
|---|---|---|
| Composition B4 | Type | by weight |
| DIISOSTEARYL MALATE | FAT | 5 |
| GLYCERYL ESTERS OF VEGETABLE | FAT | 4 |
| ISOSTEARIC, ADIPIC FATTY ACIDS | | |
| ISOPROPYL ISOSTEARATE | FAT | 23 |
| ISOSTEARYL NEOPENTANOATE | FAT | 14.5 |
| MINERAL WAX OF HYDROCARBONS | FAT | 1.75 |
| (C20/C60) | | |
| RUTILE TITANIUM DIOXIDE TREATED | COLORANT | 4.5 |
| WITH | | |
| ALUMINA/SILICA/TRIMETHYOLPROPANE (CI: 77891) | | |
| CALCIUM SALT OF RED LITHOL B | COLORANT | 0.2 |
| BLACK IRON OXIDE (CI: 77499) | COLORANT | 0.35 |
| BROWN, YELLOW IRON OXIDES (75/25) | COLORANT | 1 |
| (CI: 77491 + 77492) | | |
| POLYPHENYLTRIMETHYLSILOXY | SILICONE | 8 |
| DIMETHYLSILOXANE (VISCOSITY: 1000 | | |
| CST-MW: 3000-G/MOL) | | |
| PHENYL TRIMETHYLSILOXY TRISILOXANE | SILICONE | 4 |
| (VISCOSITY: 20 CST-MW: 372) | | |
| MICA-TITANIUM DIOXIDE (74/26) (CI: | NACRE | 2.5 |
| 77019 + 77891) (SIZE: 16-128 | | |
| MICRONS) | | |
| ETHYLENE HOMOPOLYMER | POLYMER | 3 |
| POLYETHYLENE WAX (MW: 500) | POLYMER | 6 |
| DISTEARYL DIMETHYL AMMONIUM | FILLER | 1.44 |
| MODIFIED HECTORITE | | |
| ALUMINIUM SILICATE (CI: 77004) | FILLER | 5 |
| MIXTURE OF SOYA STEROLS, | SURFACTANT | q.s. |
| ETHOXYLATED (5 EO) PROPOXYLATED (5 | | |
| PO) PENTAERYTHRITOL | | |
| Total: | | 100 |

Composition B4 comprises two nonvolatile ester oils whose viscosity is less than 15 cSt, namely isopropyl isostearate and isostearyl neopentanoate.

### Example B5

### Solid Formula Comprising at Least One Nonvolatile Ester Oil and Free from Hydrating Agent

| | | Percentage |
|---|---|---|
| Composition B5 | Type | by weight |
| MIXTURE OF PENTAERYTHRITYL | FAT | q.s. |
| ISOSTEARATE, CAPRATE, CAPRYLATE AND ADIPATE | | |
| POLYESTER OF C36 ACID DIMER AND | FAT | 4 |
| HYDROGENATED CASTOR OIL | | |
| TRIGLYCERIDE OF 2-DECYL TETRADECANOIC | FAT | 2 |
| ACID (GUERBET C24) | | |
| VIRGIN SESAME OIL | FAT | 15 |
| GLYCERYL ESTERS OF VEGETABLE | FAT | 4 |
| ISOSTEARIC, ADIPIC FATTY ACIDS | | |
| OLEYL ERUCATE | FAT | 15 |
| MICROCRYSTALLINE WAX | FAT | 8 |
| WHITE BEESWAX | FAT | 4.2 |
| MICROCRYSTALLINE WAX (C20-C60) | FAT | 2.55 |
| RUTILE TITANIUM DIOXIDE TREATED WITH | COLORANT | 4.13 |
| ALUMINA/SILICA/TRIMETHYOLPROPANE | | |
| CALCIUM SALT OF RED LITHOL B | COLORANT | 0.81 |
| ALUMINIUM LAKE OF BRILLIANT BLUE FCF | COLORANT | 0.47 |
| ON ALUMINA (12/88) | | |
| BROWN, YELLOW IRON OXIDES (75/25) | COLORANT | 1.83 |
| ETHERS OF DODECANEDIOL (2.2 MOL) AND | SURFACTANT | 8 |
| OF POLYETHYLENE GLYCOL (45 EO) | | |
| DISTEARYL DIMETHYL AMMONIUM MODIFIED | FILLER | 0.6 |
| HECTORITE | | |
| Total: | | 100 |

### Example B6

### Solid Formula Comprising at Least One Nonvolatile Ester Oil and Free from Hydrating Agent

| | | Percentage |
|---|---|---|
| COMPOSITION B6 | Type | by weight |
| OCTYLDODECYL PPG3 MYRISTYL ETHER | FAT | 11 |
| DIMER DILINOLEATE | | |
| OCTACOSANYL STEARATE | FAT | 4.25 |
| ISOCETYL STEAROYL-STEARATE | FAT | 10 |
| ISOPROPYL ISOSTEARATE | FAT | q.s. |
| ESTERS OF ACIDS AND LINEAR ALCOHOLS | FAT | 3.4 |
| (C16/C36) | | |
| MICROCRYSTALLINE WAX (C20-C60) | FAT | 2.55 |
| CALCIUM SALT OF RED LITHOL B | COLORANT | 0.064 |
| ALUMINIUM LAKE OF BRILLIANT BLUE FCF | COLORANT | 0.032 |
| ON ALUMINA (12/88) (CI: 42090:2 + | | |
| 77002) | | |
| ALUMINIUM LAKE OF BRILLIANT YELLOW | COLORANT | 0.048 |
| FCF ON ALUMINA (42/58) (CI: 15985:1 + | | |
| 77002) | | |
| MICA-TITANIUM DIOXIDE-CARMINE (CI: | NACRE | 4 |
| 77019 + 77891 + 75470) (76.5/21/2.5) | | |
| (SIZE: 28-83 MICRONS) | | |
| MICA-TITANIUM DIOXIDE-BROWN IRON | NACRE | 1 |
| OXIDE-CARMINE (59/37.5/2.5/1) (CI: | | |
| 77019 + 77891 + 77491 + 75470) | | |
| HYDROGENATED POLYISOBUTYLENE | POLYMER | 10 |
| POLYETHYLENE WAX (MW: 500) | POLYMER | 3.4 |
| VINYL ACETATE/ALLYL STEARATE | POLYMER | 7.5 |
| COPOLYMER (65/35) | | |
| POLYVINYL LAURATE | POLYMER | 8 |
| NON-HYDROGENATED POLYISOBUTYLENE (N = | POLYMER | 10 |
| 6-8-MW: 370) | | |
| ETHERS OF DODECANEDIOL (2.2 MOL) AND | SURFACTANT | 5 |
| OF POLYETHYLENE GLYCOL (45 EO) | | |
| POROUS SILICA MICROSPHERES (PARTICLE | FILLER | 5 |
| SIZE: 1-16 MICRONS) COATED WITH | | |
| POLYDIMETHYLSILOXANE (93/7) | | |
| Total: | | 100 |

### Example B7

### Liquid Formula Comprising at Least One Nonvolatile Ester Oil and Free from Hydrating Agent

| | | Percentage |
|---|---|---|
| COMPOSITION B7 | Type | by weight |
| TRIGLYCERIDE OF 2-DECYL | FAT | q.s. |
| TETRADECANOIC ACID (GUERBET C24) | | |
| TRIDECYL TRIMELLITATE | FAT | 11 |
| PENTAERYTHRITYL TETRAISOSTEARATE | FAT | 14 |
| DIISOSTEARYL MALATE | FAT | 10 |
| GLYCERYL ESTERS OF VEGETABLE | FAT | 18 |
| ISOSTEARIC, ADIPIC FATTY ACIDS | | |
| RUTILE TITANIUM DIOXIDE TREATED | COLORANT | 0.04 |
| WITH | | |
| ALUMINA/SILICA/TRIMETHYOLPROPANE (CI: 77891) | | |
| ALUMINIUM LAKE OF BRILLIANT YELLOW | COLORANT | 0.44 |
| FCF ON ALUMINA (42/58) (CI: 15985:1 + | | |
| 77002) | | |
| BLACK IRON OXIDE (CI: 77499) | COLORANT | 0.01 |
| ALUMINIUM LAKE OF DISODIUM SALT OF | COLORANT | 0.12 |
| PHLOXINE B ON ALUMINA, ALUMINIUM | | |
| BENZOATE (CI: 45410:2 + 77002) | | |
| PARTICLES OF GLASS (C-GLASS) COATED | NACRE | 0.5 |
| WITH TITANIUM DIOXIDE (AVERAGE | | |
| SIZE: 80 μM/THICKNESS: 1 μM) WITH | | |
| SILVER SHEEN | | |
| MICA-TITANIUM DIOXIDE-SILICA- | NACRE | 1 |
| TITANIUM DIOXIDE (CI: 77019 + | | |
| 77891) (PARTICLE SIZE 10-60 MICRONS) | | |
| (35/TiO₂:53/SiO₂:12) | | |
| MICA-TITANIUM DIOXIDE (77/23) (CI: | NACRE | 3 |
| 77019 + 77891) (SIZE: 16-128 | | |
| MICRONS) | | |
| MICA-TITANIUM DIOXIDE (87/13) (CI: | NACRE | 1 |
| 77019 + 77891) | | |
| POLYBUTYLENE (MONOOLEFINS/ | POLYMER | 12 |
| ISOPARAFFINS) (MW: 920) | | |
| HYDROPHOBIC PYROGENATED SILICA, | FILLER | 8 |
| SURFACE TREATED WITH DIMETHYLSILANE | | |
| (AEROSIL R 972 from EVONIK DEGUSSA) | | |
| Total: | | 100 |

### Example B8

### Solid Formula Comprising at Least One Nonvolatile Ester Oil and Free from Hydrating Agent

| | | Percentage |
|---|---|---|
| | | by |
| Composition B8 | Type | weight |
| RUTILE TITANIUM DIOXIDE TREATED WITH | COLORANT | 2.44 |
| ALUMINA/SILICA/TRIMETHYOLPROPANE (CI: | | |
| 77891) | | |
| CALCIUM SALT OF RED LITHOL B | COLORANT | 0.48 |
| ALUMINIUM LAKE OF BRILLIANT BLUE FCF ON | COLORANT | 0.14 |
| ALUMINA (12/88) (CI: 42090: 2 + 77002) | | |
| ALUMINIUM LAKE OF BRILLIANT YELLOW FCF | COLORANT | 2.29 |
| ON ALUMINA (42/58) (CI: 15985:1 + 77002) | | |
| BLACK IRON OXIDE (CI: 77499) | COLORANT | 0.22 |
| BLOCK COPOLYMER [POLYDIMETHYLSILOXANE | SILICONE | 14 |
| (DP = 100)/POLYAMIDE] | | |
| TRIMETHYL SILOXYSILICATE RESIN | SILICONE | 25 |
| MICA-TITANIUM DIOXIDE-BROWN IRON OXIDE | NACRE | 1.8 |
| (77/21/4) (SIZE: 16-128 MICRONS) | | |
| ETHYLENE HOMOPOLYMER | POLYMER | 9.2 |
| MIXTURE OF LINEAR (C30-C50) LONG-CHAIN | SURFACTANT | 2.25 |
| FATTY ALCOHOL AND OF HYDROCARBON WITH | | |
| THE SAME NUMBER OF CARBONS (80/20) | | |
| ISODODECANE | SOLVENT | q.s. |
| PURE ISOPROPYL ALCOHOL | SOLVENT | 1 |
| AMORPHOUS SILICA MICROSPHERES | FILLER | 1 |
| (GRANULOMETRY: 5 MICRONS) | | |
| SMECTITE: MODIFIED MAGNESIUM SILICATE IN | FILLER | 6 |
| ISODODECANE | | |
| Total: | | 100 |

### Example 1

### Stick of Core (A1)/Cladding (B3) Lipstick

A double-stick lipstick was made from the first composition A1 from example A1 and with the second composition B3 from example B3. The first composition was cast in a first mould so as to form a stick, with diameter of 7 mm, and was left to cool in a freezer for about an hour. The second composition was cast concentrically around the first composition after cooling and hardening of the latter in a second mould to produce a stick with diameter of 12.7 mm and the whole was left to cool in a freezer for about an hour. The first composition comprising glycerol is located in the core of the stick, and the second composition is on the outside of the stick and forms a shell around the first composition. Note that it is of course possible to envisage some other configuration or distribution of the two compositions or other proportions for the first and second compositions.

### Example 2

### Stick of Core (A1)/Cladding (B5) Lipstick and Example 3: Stick of Core (A1)/Cladding (B6) Lipstick

Another two concentric double-stick lipsticks were also made, according to the protocol described previously, by combining the first composition A1 with the second composition B5, as well as by combining the first composition A1 with the second composition B6.

### Example 4

### Liquid Lip Rouge Combining Liquid Compositions A2 and B7

A lip rouge product according to the invention was also prepared by combining each of the liquid compositions A2 and B7 in a tube, and one of the two tubes can be placed inside the other so that together, during application, the two compositions are deposited simultaneously by the applicator, for example by pressure exerted on the outer tube, and mixing of the two compositions takes place simultaneously with application on the lips.

### Example 5

### Lipstick in Core (A1)/Cladding (B8) Stick

Yet another concentric double-stick lipstick was prepared (according to the protocol described previously) by combining composition B8 with composition A1, resulting in a double-stick lipstick simultaneously providing good properties of colour, covering and stability of the colour during preparation of the lipstick, as well as good properties of comfort, durability and non-transfer.

Results from Examples 1 to 5

These various lipsticks made it possible to obtain products for making up the lips according to the invention offering good properties of durability and non-transfer, as well as good properties of comfort.

Moreover, these products according to the invention comprising two separate compositions, one of which comprises glycerol, were compared with a lipstick product that does not comprise two separate compositions but a single composition corresponding to the mixture of the first composition and of the second composition of the product according to the invention, in proportions of 50/50 by weight (i.e. as much of the first composition as of the second composition by weight in the mixture) for the various combinations envisaged for the products according to the invention described previously. The method of preparation of this comparative product is identical to that used for preparing the first and second compositions of the product according to the invention.

The results notably showed that better properties of durability and of non-transfer were obtained for the product according to the invention envisaging mixing of the two compositions simultaneously with application, relative to the comparative product.

### Example 6

### Core (A1)/Cladding (B5) Lipstick

A double-stick lipstick (core A1/cladding B5) was made from the first composition of example A1 and the second composition of example B5, according to the protocol described previously. The two compositions were cast concentrically in a mould to give sticks with a diameter of 12.7 mm and the whole was left to cool in a freezer for about an hour. The first composition comprising glycerol is located in the core of the stick (and has a diameter of 7 mm), and the second composition is on the outside of the stick and forms a shell around the first composition.

The results obtained with this double stick were compared with the results obtained with a conventional stick of diameter 12.7 mm formed solely from the composition of example 1 by application on the lips of a panel of 6 subjects with thick, clear lips:

| | | | Durability | Migration |
|---|---|---|---|---|
| | Immediate | Gloss after | of colour | after 1 |
| Product | gloss | 1 hour | (%) | hour |
| Concentric | 126.0 ±. 13.5 | 125.8 ±. 23.2 | 17 ±. 5 | 0.0 ±. 0.0 |
| Double Stick | | | | |
| comprising | | | | |
| Composition | | | | |
| A1 + | | | | |
| Composition | | | | |
| B3 | | | | |
| Stick | 119.1 ±. 13.4 | 120.4 ±. 10.6 | 17 ±. 3 | 0.0 ±. 0.0 |
| comprising | | | | |
| only | | | | |
| composition B3 | | | | |

1. A cosmetic product for making up keratinous materials, comprising:
a first composition; and
a second composition;
wherein
the first composition and second composition are applied simultaneously,
the first composition comprises:
at least one first oil phase;
at least one hydrating agent selected from polyhydric alcohols; and
less than 4 wt. % of water, relative to the weight of the composition; and
the second composition, distinct and different from the first, comprises:
at least one second oil phase; and
at least one nonvolatile ester oil; and
less than 50 wt. % of particulate phase,
relative to the weight of the composition.

2. The product according to claim 1, wherein the first composition and the second composition are solid and packaged together in one and the same packaging article.

3. The product according to claim 1, wherein the first composition and the second composition are liquid and packaged separately, in one and the same packaging article, the product comprising an applicator permitting the simultaneous application of the two compositions on the keratinous materials.

4. The product according to claim 1, wherein the hydrating agent is a polyhydric alcohol selected from the group consisting of glycerol, propylene glycol, 1,3-butylene glycol, dipropylene glycol, diglycerol, and mixtures thereof.

5. The product according to claim 1, wherein a content of the hydrating agent is from 0.001 to 30 wt. % relative to the total weight of the first composition.

6. The product according to claim 1, wherein the second composition comprises less than 3 wt. % of hydrating agent selected from the polyhydric alcohols, relative to the weight of the composition or alternatively is free from hydrating agent selected from the polyhydric alcohols.

7. The product according to claim 1, wherein the nonvolatile ester oil is a synthetic ester of formula:
R₁COOR₂
wherein
R₁ is a linear or branched fatty acid having from 1 to 40 carbon atoms and
R₂ is a hydrocarbon chain optionally branched having from 1 to 40 carbon atoms, and
a total number of carbons of R₁+R₂ is ≧11.

8. The product according to claim 1, wherein the second composition contains less than 4 wt. % of water relative to the weight of the composition.

9. The product according to claim 1, wherein the first and/or said second composition comprises at least one colorant.

10. The product according to claim 1, wherein the first composition and/or second composition comprise(s) at least one wax and/or at least one pasty fat.

11. The product according to claim 1, wherein the first composition comprises at least one nonvolatile oil and/or the second composition comprises at least one additional nonvolatile oil different from the nonvolatile ester oil.

12. The product according to claim 1, wherein the nonvolatile oil of the first composition and/or the additional nonvolatile oil of said second composition is an oil of molecular weight in the range from 650 to 10 000 g/mol.

13. The product according to claim 1, wherein a content of the nonvolatile oil or oils is 0.1 to 80% of the total weight of the first and/or of the second composition.

14. The product according to claim 1, wherein the first composition is completely free of volatile oil.

15. The product according to claim 1, wherein the product is lipstick.

16. A method of making up the skin and/or the lips, comprising:
applying the composition according to claim 1 on the skin and/or on the lips.
